(19) United States
(12) Patent Application Publication
(10) Pub. No.: US 2022/0178931 A1
(43) Pub. Date: Jun. 9, 2022

(54) Sensor

(71) Applicants: The University of Bristol, Bristol (GB); Cancer Research Technology Limited, London (GB)

(72) Inventors: Derek Neil Woolfson, Bristol (GB); William Michael Dawson, Bristol (GB); Guto Glyn Rhys, Bayreuth (DE); David Arne Scott, Bristol (GB); Jordan Michael Fletcher, Bristol (GB); Christopher Robin Wells Wood, Edinburgh, Lothian (GB); James Christopher Norman, Glasgow (GB); Cassie Jemma Clarke, Glasgow (GB)

(21) Appl. No.: 17/437,014

(22) PCT Filed: Mar. 6, 2020
(86) PCT No.: PCT/GB20/50532

§ 371 Date: Sep. 7, 2021

(30) Foreign Application Priority Data
Mar. 7, 2019 (GB) 1903054.3

(51) Int. Cl. G01N 33/574; G01N 21/64

(52) U.S. Cl. CPC G01N 33/57484; G01N 21/6428; G01N 2470/12; G01N 2800/56; G01N 2021/6439

(57) ABSTRACT

A method of diagnosing, staging or monitoring cancer, the method comprising the steps of: (a) providing a sensor array comprising at least two sensors, wherein each sensor comprises a protein barrel that comprises five or more alpha helices arranged as an alpha-helical barrel, and a reporter dye, wherein the protein barrel defines a lumen, the reporter dye is bound to the lumen reversibly; and wherein the protein barrel is different in structure in the at least two sensors; (b) contacting the sensor array with a sample obtained from a patient; and then (c) comparing the sensor array to a predetermined standard.

### CROSS REFERENCE TO RELATED APPLICATIONS

This application is a § 371 National State Application of PCT/GB2020/050532 filed Mar. 6, 2020 which claims priority to GB 1903054.3 filed Mar. 7, 2019.

### SEQUENCE LISTING

The instant application contains a Sequence Listing which has been submitted in ASCII format via EFS-Web and is hereby incorporated by reference in its entirety. The ASCII copy of the Sequence Listing, which was created on Aug. 16, 2021, is named P125296PCT—sequence listing.txt, and is 16.2 kilobytes in size.

### FIELD OF INVENTION

The present invention relates to methods involving sensor arrays and the use of sensor arrays to diagnose, stage or monitor cancer. The sensor arrays work by displacement of reporter dyes from protein barrels, and can be analysed by differential methods, often referred to as “artificial olfaction” or “artificial nose” methods.

### BACKGROUND TO THE INVENTION

There are two main approaches to sensing using biomolecules or bio-inspired molecules. The first is a “lock-and-key” approach, where a highly specific sensor molecule such as an antibody is produced for each analyte of interest. These types of sensor must be optimised to be highly selective for the target analyte, and therefore need to go through an expensive development and optimisation processes for each analyte.

A second approach is analogous to olfactory systems and uses an array of less-specific receptors. The concept is that a single target molecule or mixture binds and/or reacts with several of these receptors to different extents giving a unique signature in the array. This circumvents the need to develop the highly specific and often expensive receptors for each analyte. This second approach is referred to as differential or array sensing.

A first approach to differential sensing is where an array of dye molecules is designed and the analyte directly binds to, or chemically reacts with, the dye molecules. You, Zha and Anslyn (2015) provides a comprehensive review of such arrays and their applications. However, such arrays are complex to design because bespoke dyes must typically be designed, and these dyes must (a) provide an optical signal, (b) bind a variety of analytes, and (c) change in optical properties upon binding. Even once discovered, these bespoke dyes can involve complicated syntheses and expensive materials, increasing the cost of the final arrays.

A different approach to differential sensing is to use displacement of a reporter dye from a receptor. This allows the diversity to be engineered into the receptor rather than the dye, which enables the use of low-cost routine dyes as reporter dyes. A review of such arrays can also be found in You, Zha and Anslyn (2015). Specific representative examples are discussed below.

A commonly used receptor/dye combination is an ensemble of a short peptide, metal ion and reporter dye. The metal ion binds to both the short peptide and the reporter dye, and the analyte displaces the reporter dye from the metal ion. Umali and Anslyn (2010) describe a number of variations to this ensemble that can be used for different analyte classes. For example, in one array the peptides were decorated with guanidinium groups for binding nucleotide phosphates, and in another array the peptides were decorated with boronic acid groups for binding glycopeptides and saccharides. In more recent work, such arrays were used to characterise polyphenol compositions of wines (Umali et al., 2015) and cachaça wood extracts (Ghanem et al., 2017). These ensemble sensing arrays, however, require the careful preparation of the ensembles from at least three components, the reporter dye, the metal ion and at least one peptide. Due to the requirement for reporter dyes that bind to metal ions, and analytes that can displace the reporter dyes from binding with metal ions, such arrays also lack sensitivity towards non-polar, hydrophobic molecules.

An approach that avoids the need for a metal ion has been to use serum albumins as the receptor. Serum albumins from different source animals has been used to provide a variety of receptors, and a variety of hydrophobic dyes were used to bind within the serum albumin binding sites. These arrays have been used to discriminate between terpenes (Adams and Anslyn, 2009), fatty acids and oils (Kubarych, 2010), glycerides (Diehl et al., 2015) and the plasticisers found in different plastic explosives (Ivy et al., 2012). While useful, arrays based on serum albumins are limited to the detection and discrimination of hydrophobic molecules such as those discussed above.

The present invention seeks to provide a simple, low-cost and robust receptor and dye system that can form arrays for detecting and distinguishing between analytes, and in particular can be used in the diagnosis, staging or monitoring of cancer. As such, the present invention seeks to overcome the limitations of the prior art.

### SUMMARY OF THE INVENTION

According to a first aspect the invention provides a method of diagnosing, staging or monitoring cancer, the method comprising the steps of: (a) providing a sensor array comprising at least two sensors, wherein each sensor comprises a protein barrel that comprises five or more alpha helices arranged as an alpha-helical barrel and a reporter dye, wherein the protein barrel defines a lumen, the reporter dye is bound to the lumen reversibly, and wherein the protein barrel is different in structure in the at least two sensors; (b) contacting the sensor array with a sample obtained from a patient; and then (c) comparing the sensor array to a predetermined standard.

The inventors have realised a sensor array comprising protein barrels and a reporter dye, as described in their unpublished International Patent Application number PCT/GB2018/052521, can advantageously be used in the diagnosis, staging or monitoring of cancer.

There are currently a huge number of ways in which cancer is detected and monitored. Most of these methods can be rudimentarily sorted in to one of three groups:

(1) Biopsy. Once “suspect” tissue or growth is identified, this typically involves removing a small portion of the tumour which may involve surgery for the patient with varying degrees of invasiveness depending on the nature and location of the tumour. Once removed, suspect cells are analysed in a laboratory using an assortment of biochemical and imaging techniques. The majority of biopsies are processed as formalin-fixed paraffin-embedded (FFPE) tissues and firstly morphology is assessed by H&E (Haemotoxylin and Eosin staining). Depending on cancer type, expression of various proteins will be assessed by immunohistochemistry and/or specific stains can be used to look at specific structures or features to aid pathologists with diagnosis. Some biopsies, including breast, gastrointestinal stromal tumours, neuropathies and sarcomas, may also have fluorescent in situ hybridisation (FISH) performed to identify specific translocations or gene amplifications. Molecular pathology can also be conducted, including polymerase chain reaction (PCR) or sequencing (pyrosequencing, Sanger or Next-generation methods) to determine molecular subtypes where targeted therapies are available, or to aid diagnosis and management of the disease. This information can therefore be used to diagnose cancer type and stage, and can either confirm the growth as malignant, or alternatively, identify it as benign. The present invention can advantageously be used instead of the current techniques to determine if the cells are cancerous or benign, and even to determine what stage the cancer is at, as explained below.
(2) Scans. There are an assortment of different scanning techniques available to the medical profession when attempting to establish if a given patient has cancer-like growth that warrants further investigation. These include CT scans, nuclear medicine scan, ultrasound, MRI, PET, and X-rays. These scans offer the chance to view the location, size and distribution of a given tumour growing within a patient.
(3) Blood (or other bodily fluid) tests. These are the simplest, least invasive, and cheapest tests available and often used as a prelude to the previously mentioned tests which provide a more concrete determination of the location and size of the tumour which may be present. In short, these tests see samples (blood, urine, or faeces, etc.) collected from the patient and assayed for a marker known to be associated with the presence of cancer within the body. This can take the form of antibody detection (such as raised PSA for prostate cancer, or CA125 for ovarian cancer), complete blood counts, whole cell detection, and sequencing of circulating DNA fragments known to “leak” from tumours and enter the blood stream.

It is known that cells, including tumour cells, can secrete various substances into the blood stream (Liotta et al., 2003). These cell secreted factors are collectively referred to as the secretome (Tjalsma et al., 2000), the composition of which includes a variety of bioactive molecules ranging from proteins and lipids, to metabolites and extracellular vesicles. The components of the secretome are fundamental to cellular behaviour and physiology, playing pivotal roles in processes required for cellular proliferation, metabolism, migration and invasion. These processes are also well described hallmarks of cancer (Hanahan and Weinberg, 2000; Hanahan and Weinberg, 2011), and so the altered secretomes of cancer cells can consequently play a significant role in disease progression. Exploiting this fact, the secretomes of cancerous and non-cancerous cells have been assessed, particularly at the proteomic level, in an attempt to aid biomarker discovery that can not only infer the presence or absence of cancer, but also attempt to inform the cancer type. (Collection of relevant reviews surmised by Donadelli, 2018.). It should be noted however, that other cells in the tumour microenvironment, including cancer associated fibroblasts and immune cells, can also secrete factors that can influence disease progression. The profile of substances they secrete has been called their “secretome”. The inventors have realised that a sensor array according to the invention can advantageously be used to examine the assortment of small biological molecules circulating in healthy volunteers verses cancer patients, and the differences can be used in the invention to diagnose cancer.

It has also been discovered, that the secretome of tumour cells is different depending on whether the tumour is a primary cancer or a secondary/metastasised cancer. Remarkably this is the case, even when the tumour cells are iso-genetic, i.e. arose from the same original tumour. The inventors have surprisingly found that a sensor array as in the invention can be used to distinguish between the secretome of primary and secondary cancer cells, and therefore can be used to stage cancer. In more detail, recent work from one of the inventors has shown that tumour cells with a gain of function mutation in the tumour suppressor protein p53 have diffusible pro-invasive factor(s) in their secretomes that can influence the metastatic process (Novo et al., 2018), and they are now characterising defined secretome compartments from primary and metastatic cancer cell lines. However, data generated in support of this application has already shown that the invention has the ability to distinguish between media conditioned by primary cancer versus metastatic cancer cell lines (FIG. 17).

The inventors have also realised the potential of the sensor of the invention to offer a snapshot of an individual's health by producing a “fingerprint” for their entire biological fluid(s) (blood, urine, etc.) rather than focusing on a single biological marker in a specific fluid/sample. For a given patient this fingerprint could be tracked over time (at an annual check-up with a GP for instance) and at the first sign of the fingerprint changing and moving towards that which may suggest the presence of cancer, the patient referred to specialised treatment and scans. In this way the method of the invention can be used to monitor the onset of cancer. By testing samples from the same patient with cancer over time, the method can also be used to monitor progression of cancer from primary to secondary, or remission of cancer, and the effectiveness of a particular treatment regime.

The present invention offers many advantages to those currently being used. It has the potential to be significantly cheaper than current techniques for diagnosing, staging and monitoring cancer. It can also be less invasive, as bodily fluids can be sampled in the first instance. Samples from a biopsy can also be tested. As mentioned above, in contrast to many traditional methods, instead of testing for the presence or absence of one particular biomarker, the sensor arrays of the present invention can use differential methods to analyse complex mixtures, allowing a much more holistic approach to be taken. The present invention could be used to offer a simple and robust first pass test for cancer across the general population, with the potential to save countless lives.

### The Sensor

The reporter dye is a dye that provides a different optical signal between being bound to the lumen in the absence of any analyte and when this binding is disrupted. Disruption includes the reporter dye being ejected from the lumen or the reporter dye changing in configuration within the lumen. In the absence of an analyte, the reporter dye is bound to the lumen and produces a first optical signal. In the presence of an analyte, the reporter dye is either displaced entirely from the lumen or remains within the lumen in a different configuration, such that the signal of the reporter dye is changed.

In the present invention, the “analyte” which is being detected is present in the sample obtained from the patient.

Any individual sensor typically comprises multiple protein barrels. An analyte that results in ejection of the reporter dye from the lumen will not typically result in ejection of dye from all protein barrels within a sensor. Such an analyte will modify the dissociation constant of the reporter dye, either by direct competition with the dye or through allosteric effects, so that the equilibrium position of binding versus not binding of the reporter dye is shifted.

By using protein barrels with different structures in the different sensors, an analyte will interact with the different protein barrel structures to different extents, affecting the optical properties of the sensor to different extents, and generating an optical signal pattern across the sensor array that is specific to that analyte.

The use of alpha-helical protein barrels in displacement-based differential sensing provides a number of advantages over prior art techniques. For instance, in contrast to prior art techniques, protein barrels are not limited to detection of specific analyte classes and offer the ability to distinguish successfully a vast spectrum of target molecules and mixtures, including both hydrophobic and non-hydrophobic analytes in the context of cancer diagnosis, staging and monitoring.

One reason for this is that the structure of the protein barrel provides for a very large surface area on the lumen surface. The bound reporter dye is surrounded on all sides by the lumen surface, meaning that the chemical environment of the reporter dye is directly dictated by the large number of amino acid side chains of the lumen surface. In more detail, for an alpha-helical barrel it is common that there are up to 8 amino-acid side chains per helix that form the lumen surface, i.e. 40 amino-acid side chains for an alpha-helical barrel with five helices, 48 amino-acid side chains for an alpha-helical barrel with six helices and so on. This large surface area is advantageously provided on a rigid protein barrel where any or in theory all amino-acid side chains on the lumen surface can be modified to ultimately provide a massive variety of different protein barrels with different chemical environments. In one embodiment up to 50% of the amino-acid side chains are modified, for example 4 per chain in the lumen surface of the heptamer, so 28 in total.

Even when limiting to the 20 ribosomal, standard or proteinogenic amino acids being possible at each residue, this already provides for a massive variety in chemical environment. Therefore, the different barrels used in the sensor array may be selected from multiple millions of possible options, allowing use of protein barrels with very diverse properties. Attempting to access such variety using a protein such as serum albumin would most likely result in disruption of the tertiary structure, leading to precipitated protein with complete loss of binding ability.

Surprisingly, protein barrel sensors are not limited to analytes that can bind within the protein barrel lumen. Analyte interactions with the exterior of the protein barrel can therefore modify the environment within the protein barrel lumen, in a manner analogous to allosteric modulation of receptor binding sites found in nature. This modification can change the binding constant of the reporter dye, expelling a proportion or all of the reporter dye, or this modification can change the lumen such that the reporter dye remains bound but with different optical properties. Irrespective of the underlying reason, this effect affords the ability for the sensor array to be used on a broader spectrum of analytes than just those that can bind within the lumen. The large external surface area is again provided on a rigid protein barrel where any or all amino-acid side chains on the external surface can be modified to provide a massive variety of chemical environments

Furthermore, for each sensor the observed signal is not a simple binary signal, such as “fluorescent” or “not fluorescent”. Instead, there is a continuum between full signal and no signal. With such a massive chemical space of possible protein barrels, and with each specific protein barrel within that chemical space providing for a continuum response, the sensor assay of the invention offers access to a previously unattainable analysis space. The overall effect of this is a sensor array with an unrivalled ability to distinguish amongst a broad spectrum of analytes.

We have already noted a significant advantage of a very stable tertiary structure is that the structure can readily accommodate point mutations, particularly of residues whose side chains are directed internally within the lumen or externally toward bulk solvent. This means that the massive chemical space referred to above can feasibly be accessed without compromising the protein barrel fold. This stability of the protein barrel tertiary structure further means that it is straightforward to computationally model the structures and use rational design concepts to create an array with the desired diversity.

Another advantage of the stable and well-defined tertiary structure of a protein barrel is high reproducibility across repeat assays. The stability of protein barrels means that they remain stable over long periods of time, affording a long shelf life and/or repeated use of the sensor array, and produce the same reliable signal in response to the same analytes. Furthermore, the barrels can be freeze dried, which allows better, safer and longer storage. The barrels are then reconstituted just by adding aqueous buffer.

Protein barrels can also be produced at very low cost, either through established peptide synthesis techniques or through recombinant expression of synthetic genes. This low cost enables mass production and/or disposable sensor arrays.

The sensor array can therefore be deployed across a range of applications. For example, the sensor array can be used to identify specific compounds within complex mixtures, to differentiate between complex mixtures or to differentiate between very similar molecules, including enantiomers and enantiomeric mixtures. Specific examples discussed herein encompass the detection of a variety of both small molecules and biomolecules such as proteins. In the method of the invention the sensor array is used with samples obtained from a patient, as discussed below.

The reporter dye of the sensor array provides an optical signature, allowing for development of a sensitive but low-cost disposable chip that could be read and processed using a portable handheld device or a smartphone. In the long term, the portability of the device will facilitate ‘in line’, ‘in field’, or ‘at bedside’ analysis; in other words, bringing analysis to the problem not the problem to the lab. In particular, this technology allows for powerful yet cheap sensor devices that could be used to promote rapid and inexpensive information on the cancer of a patient.

The protein barrel comprises five or more alpha helices arranged as the alpha-helical barrel. Alpha-helical barrels are typically water soluble with a hydrophobic lumen. Both natural and de novo designed alpha-helical barrels are known, see Malashkevich et al., 1996; Koronakis et al., 2000; Zaccai et al., 2011; Fletcher, 2012; Meusch et al., 2014; Sun et al., 2014; Thomson et al., 2014; Collie, 2015; Lombardo et al., 2016 and Rhys et al., 2018. A publication by some of the inventors, Thomas et al., 2018, discloses individual alpha-helical barrels binding DPH, but not in a sensor array. Alpha-helical barrels comprise coiled-coil oligomers where the defining feature is the presence of a lumen. While coiled-coil oligomers with fewer than five alpha helices are known, five alpha helices appears to be the minimum number required to define a lumen. Alpha-helical barrels with five, six, seven, eight, ten and twelve alpha helices have been reported.

The size of alpha-helical barrels can be very precisely controlled. Controlling the lengths of the constituent alpha helices can control the length of alpha-helical barrels. Varying the number of alpha helices that make up the alpha-helical barrel can control the diameter of the lumen.

Alpha-helical barrels have a very stable tertiary/quaternary (3D) structure. Furthermore, alpha helices comprise a very predictable heptad repeat sequence. This allows for accurate modelling of the amino acid residues that form and stabilise the alpha-helical barrel 3D structure and the amino acid residues on the lumen surface and external surface of the alpha-helical barrel (as reported, for example, in Thomson et al., 2014). The stability of alpha-helical barrels also allows for the sensor array to be dried and reconstituted, washed in non-aqueous solvents and/or immobilised on a solid support.

Alpha-helical barrels are synthetically accessible. Alpha-helical barrels can comprise identical alpha helices, wherein each alpha helix comprises an identical but separate amino-acid chain. This means that only a single alpha helix needs to be synthesised, after which the alpha-helical barrel will self-assemble. This simplifies and lowers the cost of synthesising alpha-helical barrels.

The alpha helices typically comprise a sequence having a repeat unit with sequence abcdefg, wherein 50% or more of the a and d positions are hydrophobic amino acids and wherein 50% or more of the b, c, e, f and g positions are polar amino acids.

The nature of the alpha-helical heptad repeat unit typically means that the a and d positions form the lumen surface, i.e. the internal surface of the alpha-helical barrel that defines the lumen.

An important feature of alpha-helical barrels is that the rigid nature of the 3D structure allows for multiple amino acid residues to be varied simultaneously. The lumen of an alpha-helical barrel is typically hydrophobic. However, up to 50% of the amino-acid side chains facing into the lumen can be changed for any other amino acid. Even very polar or charged functional groups may be used. Due to the rigid nature of the alpha-helical barrel, the barrel can be designed so that polar functional groups can be very precisely positioned in the otherwise hydrophobic lumen without causing unfolding of the alpha-helical barrel.

With a hydrophobic lumen, the reporter dye typically should be hydrophobic. However, with polar residues in the lumen, a wider variety of dyes can be accommodated. For analytes that bind within the lumen, a similar variety of analytes can be accommodated. However, as discussed above, the analyte may also interact with the external surface of the alpha-helical barrel.

In specific embodiments the repeat unit can be selected from the list consisting of: LQKIEfI (SEQ ID NO: 1), LKAIAfE (SEQ ID NO: 2), LKEIAfS (SEQ ID NO: 3), IKEIAfS (SEQ ID NO: 4), LKEIAfA (SEQ ID NO: 5), FKEIAfA (SEQ ID NO: 6), IKEIAfA (SEQ ID NO: 7), IKEVAfA (SEQ ID NO: 8), VKEVAfA (SEQ ID NO: 9), VKEIAfA (SEQ ID NO: 10), MKEIAfA (SEQ ID NO: 11), LKQIEfI (SEQ ID NO: 12), LKEVAfA (SEQ ID NO: 13), VKELAfA (SEQ ID NO: 14), IKELSfA (SEQ ID NO: 15), IKELAfS (SEQ ID NO: 16), LKELAfS (SEQ ID NO: 17), FKEIAfA (SEQ ID NO: 18), LKQIEfI and LKELAfA (SEQ ID NO: 19); wherein f may vary between repeat units. In any given alpha helix, or in the alpha-helical barrel, up to 40%, preferably up to 25%, more preferably up to 10%, of the amino-acid residues may deviate from the repeat unit.

In a heptad repeat unit where the a and d positions form the hydrophobic core, the f position typically represents an amino acid where the side chain points directly into the bulk solvent. As such, the amino acid at the f position can vary between repeat units.

Each alpha helix can comprise at least three repeat units. Three repeat units provides for a lumen of sufficient length to bind a wide range of reporter dyes.

The entire protein barrel may comprise ribosomal, standard or proteinogenic amino acid enantiomers. Alternatively, the protein barrel can comprise non-natural amino acids. A fully enantiomeric protein barrel can form the basis for detection of enantiomeric analytes. Artificial amino acids can also be incorporated. Artificial amino acids can include natural amino acids that have been further functionalised. In one particular embodiment, the natural amino acids may have been further functionalised by post-translational modification, such as by phosphorylation or glycosylation.

The non-natural amino acid can be an amino acid that has been modified by chemically linking a protein substrate. Specifically, the protein substrate can comprise an enzyme substrate, receptor substrate and/or antibody substrate. The protein substrate may simply be for the protein binding site to bind to. The protein substrate may also be a reaction site that an enzyme can modify. For example, the protein substrate may be a phosphorylation substrate for a kinase. As such, the reporter dye signal may be affected upon binding by the kinase, or by phosphorylation.

The protein barrel can comprise a single and continuous amino-acid backbone. As such, the protein does not self-assemble from separate protein subunits. As such, the manner of self-assembly from protein subunits (i.e. quaternary structure) does not need to be considered. A single and continuous amino-acid backbone can therefore further constrain where elements of the protein secondary structure become located in the fold. With alpha-helical barrels, for example, each helix may have a different structure, or just one helix of the barrel may contain a charged residue. With separate alpha helix subunits, consideration would need to be given to the different permutations of helical barrels that could form. With a single and continuous amino acid backbone, this consideration can be largely removed by careful design of a single and continuous amino acid backbone that folds into the alpha helices (i.e. the secondary structure) that in turn folds into the alpha helix barrel (i.e. the tertiary/quaternary structure).

Overall, significant control over making specific changes to a protein barrel structure can be gained.

The protein barrel can be in solution, but in one embodiment of the invention the protein barrel is immobilised on a substrate. This allows for sensor arrays where analyte solutions can flow over the sensors, or where sensors can be washed and used again. Furthermore, immobilisation provides for sensor arrays where there are no physical barriers between sensors, providing the basis for array microchips. The amounts of protein barrel needed for such array microchips would be miniscule, probably less than one microgram, such as 0.01 to 1 microgram.

The reporter dye can also be immobilised on the substrate, or on the protein barrel, as long as the reporter dye is still able to reversibly access the protein barrel lumen. Such immobilisation provides for protein barrels and reporter dyes that cannot wash away or interfere with neighbouring sensors, and provides for reusable sensor arrays or sensor arrays that can be used for in-line sensing.

The protein barrel can also be situated on or in a hydrogel, or 3-dimensional porous scaffolds. This helps to allow the barrel to be used for sensing gaseous analytes that can dissolve in the hydrogel and become accessible to the barrel.

The protein barrel and reporter dye can be in a dry state. In other words, the complex of the protein barrel and reporter dye has been dried. The sensor array is therefore in a dry state. The dry state is suitable for storage, but would typically be rehydrated before carrying out analysis. If the analyte is aqueous, rehydration could be achieved in a simple manner by the analyte. The use of a dry state is made possible by the protein barrels being highly stable.

In a preferred embodiment, the reporter dye provides an optical signal when bound to the lumen. By this, we mean that there is a measurable optical signal when the reporter dye is bound to the lumen. Typically, this would mean that there is no optical signal when the reporter dye is in free solution. This has advantages over the inverse scenario, where the reporter dye provides a signal in free solution but provides no signal when bound to the lumen, but the inverse scenario is also possible.

The resting state of the reporter being bound to the protein barrel, before any analyte is added, is a state where a positive signal can be measured. This provides a quick way of checking that the reporter dye and protein barrel in each sensor are intact before starting the assay. In addition, it is postulated that in certain cases the reporter dye may not leave the lumen in response to an analyte. The reporter dye instead adopts a different configuration within the lumen, possibly in response to a change in the lumen configuration, this change in configuration also causing a change in optical properties. If the reporter dye was quenched on being bound, such changes would not be observable. This furthermore allows for a reporter dye to be encapsulated within the lumen, perhaps by appending blocking groups on either end of the lumen after the reporter dye is bound. Such a complex would operate by changes in configuration of the reporter dye within the lumen in response to target analytes. Encapsulating reporter dyes in this way allows for robust sensors that can be reused, or used in applications such as in line sensors, as the reporter dye would not wash away.

The reporter dye can be a compound according to Formula I

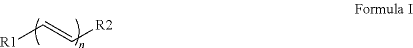

wherein n is 3 or more, preferably n is 3, 4 or 5, more preferably n is 3; and R1 and R2 are independently selected from aryl or heteroaryl, preferably aryl, more preferably phenyl. Preferably, the reporter dye is 1,6-diphenyl-1,3,5-hexatriene. Reporter dyes such as these are long, thin and hydrophobic, which means they are well suited to binding within a protein barrel lumen. Moreover, reporter dyes such as these do not provide an optical signal in free solution. However, on binding to a protein barrel lumen, the unconjugated chain twists and can provide a fluorescent signal in response to ultraviolet light.

The sensor array can comprise at least 10 sensors, preferably at least 50 sensors, more preferably at least 100 sensors, yet more preferably at least 300 sensors, wherein the protein barrel is different in each of the at least 10, 50, 100 or 300 sensors respectively. It is predicted that about 16 sensors, each with a different protein barrel, would be required to detect most commercially relevant small and macromolecular analytes. Of course, flatbed plate readers are typically set up to read 96-, 384- and 1536-well plates, although controls and replicates will usually bring down the number of unique sensors in any plate.

The sensor array can comprise at least one further sensor, wherein the reporter dye is different in the at least one further sensor. Varying the reporter dye is another way to achieve a variation in signal across the array. By using a reporter dye with different physicochemical properties to those of Formula I, the ability to distinguish different analytes is further improved. Typical reporter dyes that can be used include all napthalene dyes, such as 6-propionyl-2-dimethylaminonaphthalene (prodan).

The sensor array can be incorporated into a microarray chip. As mentioned above, it is possible to fabricate a microarray chip using the protein barrel and reporter dye. This can be a low cost, disposable or reusable microarray with a powerful ability to identify a broad range of analytes. The microarray can be read by a smartphone, making this sensor technology available for use by the population at large.

### The Method

In the method of the invention, the sensor array is contacted with a sample obtained from a patient. The sample will usually be in liquid form, and will contain biological material.

For example, samples obtained directly from the patient can be any suitable bodily fluids or materials, including for example whole blood, plasma, serum, cerebrospinal fluid, saliva, semen, sputum, urine or stool. These samples will contain secretome from cells in the body that may be cancerous or non-cancerous, and so can be used to analyse that secretome. Bodily fluids can be used directly in the sensors arrays of the present invention, or can be treated by filtering or centrifuging to remove any particulate matter that may be present. Any other treatment to make the sample obtained from the body more suitable for analysis in the sensor array, such as dilution, can be used.

In another embodiment, a sample is obtained from a patient and then used indirectly, in the sense that cells or other biological material will be collected from the patient and used to obtain a liquid sample. The biological material can be obtained from any of the source from the patient, including a cell scraping, a biopsy tissue, or bone marrow.

Often the biological material will be from a biopsied tumour or tissue which may or may not be cancerous. Often the sample will be a liquid in which the cells obtained from the patient have been cultured, also known as the supernatant, or “conditioned media”.

The patient is preferably a mammal, especially a primate. In one embodiment, the patient is a human.

Following contact of the sample with the sensor array, the sensor array is then compared to a pre-determined standard. This will depend on whether the method is for diagnosing, staging or monitoring cancer, as follows. The comparison to a predetermined standard can comprise the use of computational pattern recognition, such as those implemented using machine learning, other or relates Artificial Intelligence methods. A person skilled in the art can easily use available techniques to develop methods for analysing the optical fingerprints from the sensor arrays.

In more detail, as explained below, the invention works by exploiting the fact that the secretome of healthy cells differs from cancerous cells. Furthermore, the secretome produced by different cancerous cells also differs, for example between different types of cancer, and between primary and secondary cancer cells. The sample will contain the secretome of cells obtained from the patient's body. The sensor array can detect these difference between these secretomes even when present as part of complex mixture. Each secretome gives rise to a different optical signal in the assay yielding a unique fingerprint. In the invention, molecules that form part of the secretome are the “analytes” for the sensor array, along with the rest of the biological fluid in the sample.

It is envisaged that all types of cancer could be detected using the sensor arrays of the present invention. Using samples collected from patients with cancer, as well as healthy volunteers, a set of standards can be collected. Using standard computer-based machine learning techniques, a skilled person would be able to develop the ability to distinguish between samples collected from patients with or without cancer. A skilled person would also be able to distinguish between samples collected from patients with different sorts of cancer and/or stages of cancer. A skilled person would also be able to distinguish between patients with primary or secondary tumours.

The cancer in question could include solid cancer tumours, including, but not limited to, breast, pancreatic adenocarcinoma, colorectal carcinoma, renal, endometrial, ovarian, thyroid, and non-small cell lung carcinoma, melanoma, prostate carcinoma, sarcoma, gastric cancer and uveal melanoma; and liquid tumours, including but not limited to, leukaemias (particularly myeloid leukaemia) and lymphomas. The present invention is particularly useful for diagnosing breast cancer, especially breast cancer as a primary cancer, and metastatic breast cancer, particularly where the metastasis cancer is in the lung.

It is envisaged that for a given patient a sample will be obtained (or provided) either directly or indirectly. The sample will be assayed using the sensor array. This will generate a fluorescent fingerprint specific for that given sample. Computer-aided machine-learning, pattern-recognition, or related AI or other techniques could be used to determine if the fingerprint produced by the patient's sample is most similar to fingerprints produced by standard samples collected from patients with cancer or from healthy volunteers. A score would be provided to gauge the similarity of the patient's fingerprint to that seen in healthy or cancerous standards.

The present invention can also be used to identify the nature and type of cancer that may be present in a given patient as it has been found that various cancer types produce a different secretome.

For a patient identified as having cancer, computer-aided machine-learning, pattern-recognition, related AI or other techniques could be used to analyse the fingerprint produced by the patient's sample and compare it against a set of standards (obtained from patients known to have a specific type of cancer) to determine which type of cancer the fingerprint is most indicative of.

Furthermore, for a patient identified as having cancer, computer-aided machine-learning, pattern-recognition, related AI or other techniques could be used to analyse the fingerprint produced by the patient's sample and compare it against a set of standard fingerprints produced by patients with either primary tumour or secondary tumour. The similarity of the patient's fingerprint to either of these two classes will suggest if the tumour is of primary or secondary origin.

The present invention can also be used to monitor cancer. For example, to determine if a patient is responding to treatment intended to reduce and/or eliminate the cancer from the patient's body. In this application samples would be collected over the course of their treatment. The sensor array would be used to generate a set of fingerprints over time. Each fingerprint would be examined using computer-aided machine learning and pattern recognition techniques. At each step, the similarity of the fingerprint to healthy or cancerous standards would be determined. An increase in similarity between the patient's fingerprint and to those generated from healthy volunteers would indicate treatment was being effective.

The present invention can also be used as part of a routine check-up and routine health monitoring. As part of a periodic check-up, likely conducted by a GP, samples will be collected and analysed using the sensor array. Fingerprints generated for the specific patient, using a specific sample type (such as blood, serum, or urine etc) would be collected over time. This would establish a baseline fingerprint specific for the patient. Each fingerprint would be examined using computer-aided machine-learning, pattern-recognition, related AI or other techniques to monitor any changes in the fingerprint and to examine any increase in similarity towards fingerprints generated by patients known to have cancer.

As part of the present invention samples will be obtained from patients with an assortment of cancer types, including those originating in different tissues or regions of the body, and of primary of secondary origin. Samples will also be obtained from healthy volunteers. These samples will be analysed using the sensor array. This will generate a fingerprint for each sample. These fingerprints will be used to train machine-learning, related or other algorithms. Fingerprints will be combined in sets to train specific algorithms for given prediction applications. For example, for a simple prediction of whether a given patient has cancer or not, all fingerprints obtained from patients with cancer are combined into a single set. Similarly, all fingerprints from healthy volunteers are combined in to a single set. These two sets are then used to train algorithms. These two sets of fingerprints effectively serve as standards against which a given patient's fingerprint will be compared (akin to a database). Machine-leaning, related AI and algorithms could be used to classify a given patients fingerprint as being most similar to cancerous or non-cancerous standards.

For other prediction and classification applications, fingerprints will be pooled in to sets for training machine leaning algorithms as appropriate. For example, for primary vs. metastatic tumour burden classification, or by cancer type.

In embodiments of the invention, a patient diagnosed as having cancer using the method of the invention, may then be treated for cancer, for example by chemotherapy, radiotherapy, immunotherapy and/or surgery.

### BRIEF DESCRIPTION OF THE DRAWINGS

FIG. 1 is a schematic showing the abcdefg heptad repeat units of two alpha helices in a coiled-coil arrangement;

FIG. 2 is a schematic showing the abcdefg heptad repeat units of five or more alpha helices in a coiled-coil alpha-helical barrel;

FIG. 3 is a schematic showing the abcdefg heptad repeat units of a six-helix barrel;

FIG. 4 shows top-down and side views of x-ray crystal structures of coiled-coil folds comprising 3, 4, 5, 6 and 7 alpha helices, corresponding to PDP IDs 4DZM, 4DZL, 3R4A, 4PN8, 4PN9 and 4PNA, respectively;

FIG. 5A shows a partial cutaway view of an x-ray crystal structure of an alpha-helical barrel comprising CC-Hex2 with farnesol bound in the alpha-helical barrel lumen;

FIG. 5B shows a top down view of the x-ray crystal structure of FIG. 5A;

FIG. 6 shows a sensor array of different alpha-helical barrels;

FIG. 7 is a schematic view showing how a sensor array is run, wherein protein barrels are added to the array, DPH reporter dye is bound and different analytes produce different displacement patterns or fingerprints;

FIG. 8 shows displacement patterns for seven different analytes against the alpha-helical barrel array described in FIG. 6;

FIG. 9 shows replicate displacement patterns for cholesterol;

FIG. 10 shows a process for analysing a displacement pattern using computational methods;

FIG. 11 is a chart showing how computational pattern recognition improves with training;

FIG. 12 shows the DPH displacement fingerprints produced by selected tea samples demonstrating that complex mixtures can be successfully analysed;

FIG. 13 shows across the top the DPH displacement fingerprints for glucose, galactose and mannose and across the bottom the structure of the epimers, and demonstrates that the invention can be used to distinguish epimers;

FIG. 14 is a comparison of DPH displacement fingerprints for cholesterol (right), at 1 μM final concentration, using proteinogenic (left) and non-proteinogenic (centre) peptide arrays;

FIG. 15 shows DPH displacement fingerprints for N-Acetyl-L-aspartic acid (Panel A) and NG,NG-Dimethylarginine (Panel B) using a peptide barrel array including one all D-amino acid peptide (d-(avkeva)) which is represented by the block depicted on the left, second from bottom in each fingerprint;

FIG. 16 shows the fingerprints generated for all 10 conditioned media samples. This includes media collected from cells of “non-cancerous”, “primary tumour”, and “metastasised tumour” origins as indicated. Each fingerprint conditioned media is labelled as follows. A: NMuMg; B: HC11; C:EpH4; D: Yej; E: 113; F: 724; G: Yej-M1; H: Yej-M2; I: 113-M1; J: 113-M2;

FIG. 17 shows the fingerprints generated for combined “non-cancerous” (panel A), “primary tumour” (B) and metastasised tumour (C) derived conditioned media;

FIG. 18 is the confusion matrix for 2-way prediction of healthy, non-cancerous cells, and cells originating tumours;

FIG. 19 is the confusion matrix for 3-way prediction of healthy, non-cancerous cells, and cells originating from primary tumours and metastatic tumours; and

FIG. 20 is the confusion matrix for 2-way prediction of cells originating from primary tumours and metastatic tumours.

### DESCRIPTION

The first aspect of the invention involves providing a sensor array comprising at least two sensors. The sensor array can be provided, for example, in a multiwell plate. In this case, the different sensors would be in different wells.

The sensor array comprises at least two sensors. Two sensors is the minimum number of sensors needed to define an array. A larger number of sensors can be included in the array. For example, the array can comprise at least 10 sensors, preferably at least 50 sensors, more preferably at least 100 sensors, yet more preferably at least 300 sensors. The protein barrel is different in each of the at least two, 10, 50, 100 or 300 sensors respectively.

The requirement for the protein barrel to be different in structure in the claimed sensors does not preclude that the sensor array can contain yet further sensors that are merely replicate sensors, controls, or make use of the same protein barrel but with a different reporter dye. Indeed, the use of replicate sensors is a common strategy to improve data quality. In other words, the sensor array comprises a number of different sensors with different protein barrels, but there will usually be further sensors in the sensor array with the same protein barrels. These further sensors are usually replicates for data quality, controls, or sensors that use a different reporter dye. However, within the sensor array, there must at least be the claimed number of sensors wherein the protein barrel is different in structure.

Each sensor comprises a protein barrel. A protein barrel is a protein that defines a lumen. The protein barrel therefore has a lumen surface and an external surface. A lumen is a tubular cavity within the protein. The tubular cavity is typically elongated, i.e. long and narrow. Usually, the lumen would be open at both ends to allow for displacement of molecules within the lumen. However, in certain embodiments, the lumen may be blocked at one or at both ends to trap specific molecules within the lumen.

The protein barrel is different in structure in the different sensors. By this, we mean that there is at least one difference by which the protein barrels can be distinguished. This difference could include a point mutation in an amino acid, or an amino acid that has been derivatised or functionalised. This difference could also include a change in length or width of the protein barrel. This difference could also include a change in type of protein barrel.

Due to the possibility of making very different chemical environments by using a limited number of differences in the protein backbone, in certain embodiments of the invention, the different protein barrels may have similar protein backbones. For example, the different protein barrels may all be of the same type. In one embodiment, the different protein barrels may all be alpha-helical barrels. In another embodiment, it may be that the different protein barrels are within 50% sequence identity, 70% sequence identity or 90% sequence identity.

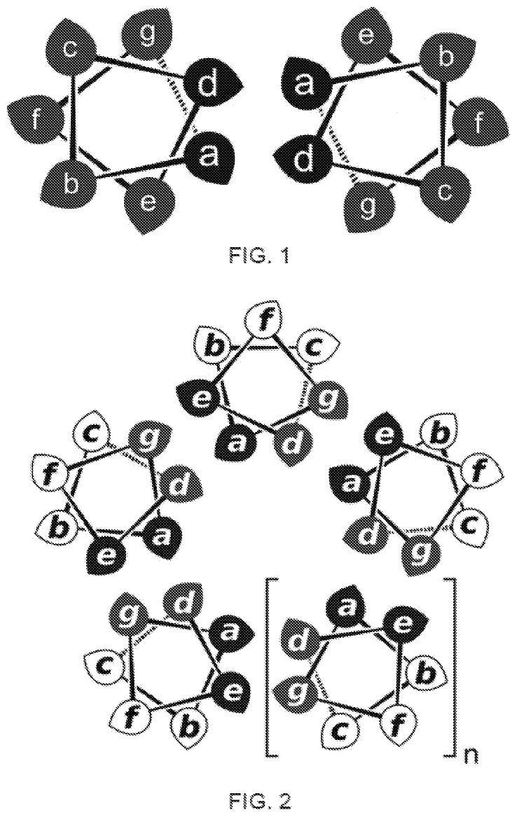

Alpha-helical barrels are protein barrels that comprise five or more alpha helices. The alpha helices arrange in a pattern where they are substantially aligned with each other, side-by-side, to form a tube-like shape. This is known as a coiled-coil fold (also known as coiled-coil structures or assemblies) and has been well characterised previously. Representative examples include Malashkevich et al., 1996; Koronakis et al., 2000; Zaccai et al., 2011; Fletcher et al., 2012; Meusch et al., 2014; Sun et al., 2014; Thomson et al., 2014; Collie et al., 2015; and Lombardo et al., 2016. Examples of coiled-coil folds comprising different alpha helix numbers can be seen in FIGS. 1-4.

As can be seen in FIG. 4, coiled-coil folds can occur with 3 and 4 alpha helices. However, it is not until the number of alpha helices reaches 5 that a lumen forms. Coiled-coils with 5 or more alpha helices form a lumen, and therefore constitute alpha-helical barrels.

Thomson 2014 reports that five alpha-helix barrels have a lumen diameter of about 5.7 Å, six alpha-helical barrels have a lumen diameter of about 6.0 Å or about 7.4 Å, and seven alpha-helical barrels have a lumen diameter of about 7.6 Å, as measured by x-ray crystallography. In certain embodiments, the protein barrels have a lumen diameter of greater than about 5 Å, more preferably more than about 5.5 Å. In certain embodiments, the protein barrels have a lumen diameter of less than about 10 Å, more preferably less than about 8 Å.

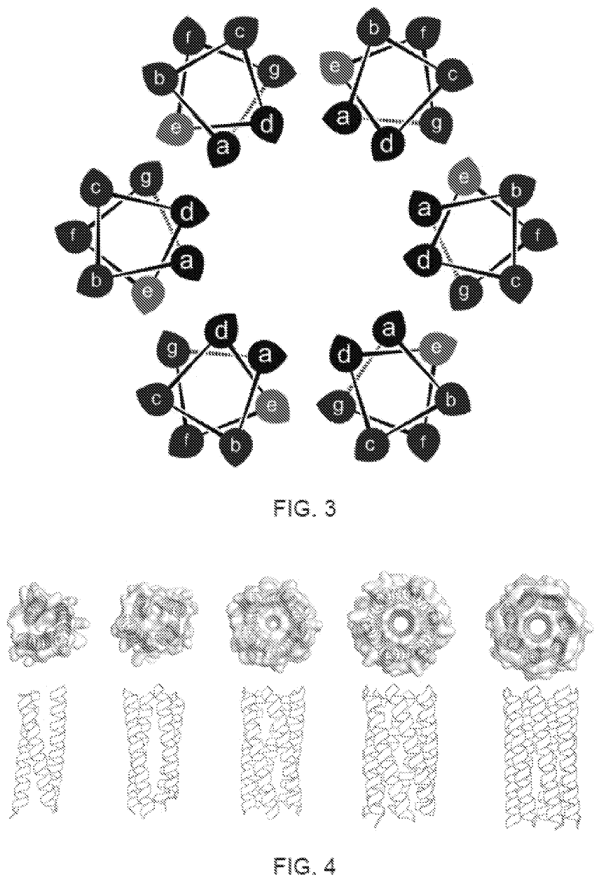

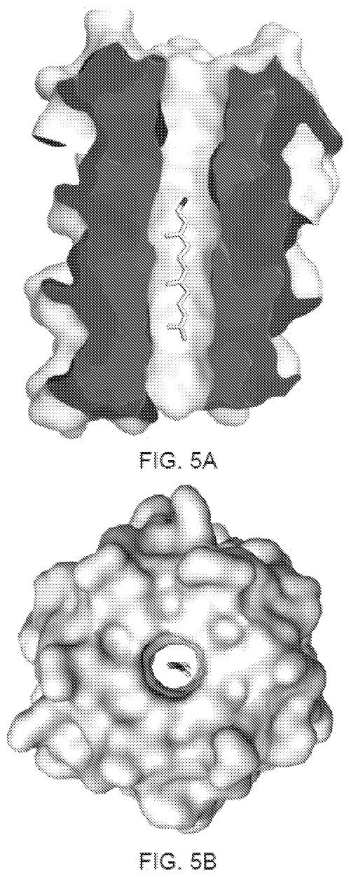

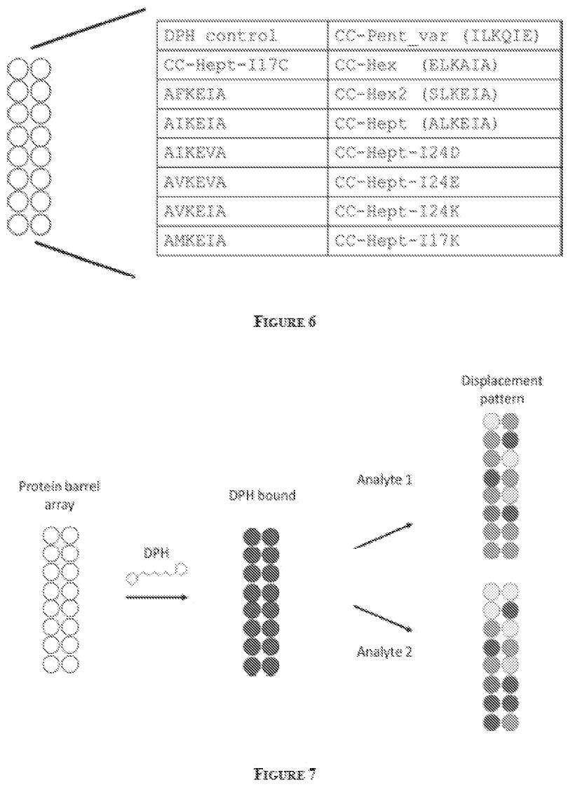

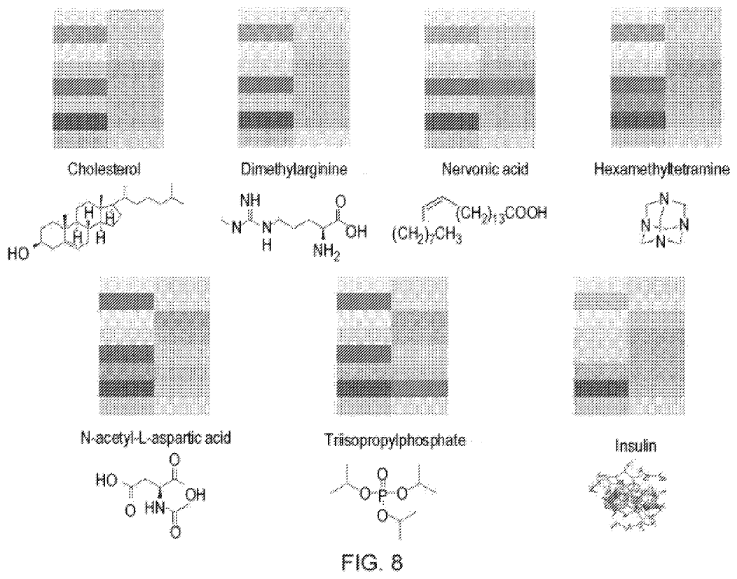

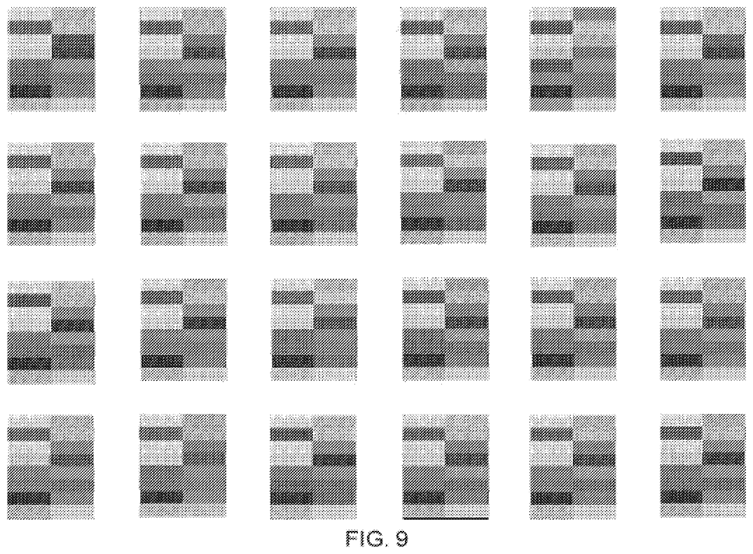

A common structural feature in coiled-coil folds, such as in alpha-helical barrels, is that each alpha helix can independently comprise a sequence having a repeat unit with sequence abcdefg, wherein 50% or more of the a and d positions are hydrophobic amino acids and wherein 50% or more of the b, c, e, f and g positions are polar amino acids. In particular, having hydrophobic amino acids at the e and g positions can encourage alpha helix barrel formation, as can be seen in FIGS. 2 and 3. In one example, all the b, c and f positions can be polar amino acids, while all e and/or all g positions are hydrophobic amino acids.

In further embodiments, 60% or more, 75% or more, or 90% or more of the a and d positions are hydrophobic amino acids. In yet further embodiments, 60% or more, 75% or more, or 80% or more of the b, c, e, f and g positions are polar amino acids.

In particular examples, the repeat unit with sequence abcdefg can be selected from the list consisting of: LQKIEfI (SEQ ID NO: 1), LKAIAfE (SEQ ID NO: 2), LKEIAfS (SEQ ID NO: 3), IKEIAfS (SEQ ID NO: 4), LKEIAfA (SEQ ID NO: 5), FKEIAfA (SEQ ID NO: 6), IKEIAfA (SEQ ID NO: 7), IKEVAfA (SEQ ID NO: 8), VKEVAfA (SEQ ID NO: 9), VKEIAfA (SEQ ID NO: 10), MKEIAfA (SEQ ID NO: 11), LKQIEfI (SEQ ID NO: 12), LKEVAfA (SEQ ID NO: 13), VKELAfA (SEQ ID NO: 14), IKELSfA (SEQ ID NO: 15), IKELAfS (SEQ ID NO: 16), LKELAfS (SEQ ID NO: 17), FKEIAfA (SEQ ID NO: 18), LKQIEfI and LKELAfA (SEQ ID NO: 19); wherein f may vary between repeat units. While these repeat units represent the basic building block of an alpha helix, there may of course be point mutations such that not every unit is an identical repeat. In any given alpha helix, or in the alpha-helical barrel, up to 40%, preferably 25%, more preferably 10%, of the amino acid residues may deviate from the repeat unit. It can be seen from FIGS. 2 and 3 that position f is directed towards the bulk solvent and plays little role in assembly of the alpha helices with each other. The amino-acid residue at position f is therefore less important, and can vary between repeat units. Position f is therefore usually a polar amino acid to assist with water solubility of the alpha-helical barrel. However, position f is also a good candidate for further functionalisation.

Each alpha helix can comprise at least three repeat units. Examples of full-length sequences based on the above repeat units include the following.

| | Sequence | |
|---|---|---|
| Peptide Name | cdefgabcdefgabcdefgabcdefgab | |
| CC-Pent | Ac-GKIEQILQKIEKILQKI | (SEQ ID |
| | EWILQKIEQILQG-NH2 | NO: 20) |
| CC-Hex | Ac-GELKAIAQELKAIAKEL | (SEQ ID |
| | KAIAWELKAIAQG-NH2 | NO: 21) |
| CC-Hex2 | Ac-GEIAKSLKEIAKSLKEI | (SEQ ID |
| | AWSLKEIAKSLKG-NH2 | NO: 22) |
| CC-Hept | Ac-GEIAQALREIAKALREI | (SEQ ID |
| | AWALREIAQALRG-NH2 | NO: 23) |
| CC-Hex2-I10K | Ac-GEIAKSLKEKAKSLKEI | (SEQ ID |
| | AWSLKEIAKSLKG-NH2 | NO: 24) |
| CC-Hept-I17K | Ac-GEIAQALREIAKALREK | (SEQ ID |
| | AWALREIAQALRG-NH2 | NO: 25) |
| CC-Hept-I24D | Ac-GEIAKALREIAKALREI | (SEQ ID |
| | AWALREDAKALRG-NH2 | NO: 26) |
| CC-Hept-I24K | Ac-GEIAQALREIAKALREI | (SEQ ID |
| | AWALREKAQALRG-NH2 | NO: 27) |
| CC-Hept-I24E | Ac-GEIAKALREIAKALREI | (SEQ ID |
| | AWALREEAKALRG-NH2 | NO: 28) |
| AIKEVA | Ac-GEVAQAIKEVAKAIKEV | (SEQ ID |
| | AWAIKEVAQAIKG-NH2 | NO: 29) |
| AIKEIA | Ac-GEIAQAIKEIAKAIKEI | (SEQ ID |
| | AWAIKEIAQAIKG-NH2 | NO: 30) |
| AVKEIA | Ac-GEIAQAVKEIAKAVKEI | (SEQ ID |
| | AWAVKEIAQAVKG-NH2 | NO: 31) |
| AVKEVA | Ac-GEVAQAVKEVAKAVKEV | (SEQ ID |
| | AWAVKEVAQAVKG-NH2 | NO: 32) |
| ALKEVA | Ac-GEVAQALKEVAKALKEV | (SEQ ID |
| | AWALKEVAQALKG-NH2 | NO: 33) |
| AVKELA | Ac-GELAQAVKELAKAVKEL | (SEQ ID |
| | AWAVKELAQAVKG-NH2 | NO: 34) |
| SIKELA | Ac-GELAQSIKELAKSIKEL | (SEQ ID |
| | AWSIKELAQSIKG-NH2 | NO: 35) |
| AIKELS | Ac-GELSQAIKELSKAIKEL | (SEQ ID |
| | SWAIKELSQAIKG-NH2 | NO: 36) |
| SIKELA | Ac-GELAQSIKELAKSIKEE | (SEQ ID |
| | AWSIKELAQSIKG-NH2 | NO: 37) |
| ALKELA | Ac-GELAQALKELAKALKEL | (SEQ ID |
| | AWALKELAQALKG-NH2 | NO: 38) |
| SLKELA | Ac-GELAQSLKELAKSLKEL | (SEQ ID |
| | AWSLKELAQSLKG-NH2 | NO: 39) |
| ALKELA | Ac-GELAQALKELAKALKEQ | (SEQ ID |
| | AWALKELAQALKG-NH2 | NO: 40) |
| ALKELA | Ac-GELAQALKELAKALKEE | (SEQ ID |
| | AWALKELAQALKG-NH2 | NO: 41) |
| AFKEIA | Ac-GEIAQAFKEIAKAFKEI | (SEQ ID |
| | AWAFKEIAQAFKG-NH2 | NO: 42) |
| AMKEIA | Ac-GEIAQAMKEIAKAMKEI | (SEQ ID |
| | AWAMKEIAQAMKG-NH2 | NO: 43) |
| CCHept- | Ac-GEIAQALKEIAKALKEC | (SEQ ID |
| I17C | AWALKEIAQALKG-NH2 | NO: 44) |
| CCPent_var | Ac-GQIEQILKQIEKILKQI | |
| | EWILKQIEQILKG-NH₂ | |

CC-Pent, CC-Hex2, CC-Hept and AIKEIA point mutants where the b (or c in CC-Pent) position is either K or R, and the f positions are either QKWQ or KKWK, and the mutation is at the 3, 7, 10, 14, 17, 21, 24, 28 position:

CC-Pent-Mutants: Ac-GcIEfILQcIEfILQcIEfILQcIEfILQG-NH₂

CC-Hex2-Mutants: Ac-GEIAfSLbEIAfSLbEIAfSLbEIAfSLbG-NH₂

CCHept-Mutants: Ac-GEIAfALbEIAfALbEIAfALbEIAfALbG-NH₂

AIKEIA-Mutants: Ac-GEIAfAIbEIAfAIbEIAfAIbEIAfAIbG-NH₂

Each alpha helix listed above is not covalently linked to any other alpha helices within the fully formed alpha-helical barrel. Instead, the alpha helices self-assemble. The alpha-helical barrels formed from the peptides listed above comprise identical alpha helices. However, in different embodiments, the alpha helices within an alpha-helical barrel can be non-identical. With non-identical alpha helices that are not covalently linked, attention should be paid to the different permutations of alpha-helical barrels that can self-assemble. Alternatively, the alpha-helical barrel can comprise a single and continuous amino acid backbone. This affords a much greater level of control over the alpha helices that assemble to form the alpha-helical barrel.

The protein barrel can comprise a non-natural amino acid. This may be an enantiomer of a natural amino acid, a natural amino acid that has been further functionalised, or any other amino acid. The rigid structure of protein barrels generally allows for substitution of a number of amino acids without compromising the fold of the protein barrel.

For example, the table below shows how 3 non-proteinogenic peptides are incorporated into the array of 15 barrels and a DPH control by replacing 3 proteinogenic peptides.

Proteinogenic array

| | |
|---|---|
| DPH control | CC-Pent_var (ILKQIE) |
| CC-Hept-I17C | CC-Hex (ELKAIA) |
| AFKEIA | CC-Hex2 (SLKEIA) |
| AIKEIA | CC-Hept (ALKEIA) |
| AIKEVA | CC-Hept-I24D |
| AVKEVA | CC-Hept-I24E |
| AVKEIA | CC-Hept-I24K |
| AMKEIA | CC-Hept-I17K |

Non-proteinogenic array

| | |
|---|---|
| DPH control | CC-Pent_var (ILKQIE) |
| CC-Hept-I17C | CC-Hex (ELKAIA) |
| AFKEIA | CC-Hex2 (SLKEIA) |
| AIKEIA | CC- Hept (ALKEIA) |
| AIKEVA | CC-Hept-124D |
| AVKEVA | CC-Hex-L24E |
| AVKEIA | CC-Hept-L28NIE |
| AMKEIA | CC-Hept-dL (AdLKEIA) |

As can be seen, peptides in the standard proteinogenic array are shown on the left and the non-proteinogenic array on the right incorporates 3 peptide sequences with unnatural amino acids. Nle=Norleucine, dL=Dehydroleucine.

CCHept-L28Nle:
Ac-GEIAQALKEIAKALKEIAWALKEIAQANleKG-NH2
CCHept-dL:
Ac-GEIAQAdLKEIAKAdLKEIAWAdLKEIAQAdLKG-NH2
CCHex-L24Nle:
(SEQ ID NO: 46)
Ac-GELKAIAQELKAIAKELKAIAWENleKAIAQG-NH2

In one embodiment, the non-natural amino acid is an amino acid that has been modified by chemically linking a protein substrate. Such methods of chemical linkage are well known. The protein substrate would typically be linked to a residue on the external surface of the protein barrel. Where an alpha-helical barrel is used, position f of the heptad repeat on an alpha helix would be a suitable candidate for the anchor for the linker. The protein substrate can comprise an enzyme substrate, receptor substrate and/or antibody substrate. By providing a protein substrate, the target protein can bind to the protein barrel and/or chemically modify the protein substrate. Either the binding of the protein or the chemical modification of the protein substrate can change the configuration of the protein barrel lumen and, in turn, disrupt binding of the reporter dye.

Each sensor of the sensor array comprises a reporter dye. A dye is a molecule that can provide an optical signal. The optical signal is typically in the ultraviolet and/or visible spectrum. By this, we mean a molecule that can provide a signal in the ultraviolet-visible region of the electromagnetic spectrum. The optical signal may be an absorption or luminescence signal. Preferably, the optical signal is fluorescence.

In the sensor array, the reporter dye is bound to the lumen reversibly. By this, we mean that the reporter dye is bound entirely, or substantially, within the protein barrel lumen. The binding is reversible, meaning that the reporter dye is free to unbind from the lumen, or to undergo changes in binding within the lumen. This reversible binding is typically mediated by non-covalent interactions. A particularly preferable form of reversible binding is mediated by a hydrophobic reporter dye binding within a hydrophobic lumen. Labile covalent binding may also be used, for example, by means of an imine that can be readily cleaved by nucleophilic substitution.

To qualify as a reporter dye, the molecule should provide a different signal between being bound to the lumen and when this binding is disrupted. Disruption includes the reporter dye being ejected from the lumen or the reporter dye changing in configuration within the lumen. Ejection may occur when an analyte enters the lumen and displaces the reporter dye, in other words, by competitive binding. Ejection may also occur when an analyte binds to the exterior of a protein barrel such that the lumen changes in configuration to the extent that the reporter dye can no longer bind to the lumen. Alternatively, in this scenario, the change in configuration of the lumen results in a change in configuration of the reporter dye.

The reporter dye can be free to leave the lumen, for example, when the lumen is open at both ends. In an alternative embodiment, the reporter dye is encapsulated within the lumen. In this embodiment, the sensor relies on an analyte changing the lumen configuration such that the reporter molecule changes in configuration and exhibits a different signal.

In a preferred embodiment, the reporter dye provides an optical signal when bound to the lumen. For reporter dyes that can provide signals constituting a positive signal or no signal, depending on environment, (for example, a reporter dye that can fluoresce in one environment but cannot fluoresce in a different environment), the positive signal exists when the reporter dye is bound to the lumen. This is in contrast to a reporter dye where the optical signal exists in free solution, but does not exist when bound to the protein lumen.

The reporter dye can be a compound according to Formula I

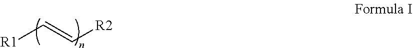

wherein n is 3 or more, preferably n is 3, 4 or 5, more preferably n is 3; and R1 and R2 are independently selected from aryl or heteroaryl, preferably aryl, more preferably phenyl. Reporter dyes in accordance with Formula I are therefore generally hydrophobic and able to adopt an elongate configuration. In a preferred embodiment, the dye is 1,6-diphenyl-1,3,5-hexatriene.

Alternative dyes may be used, including any naphthalene such as 6-propionyl-2-dimethylaminonaphthalene (prodan).

The sensor array may comprise at least one further sensor, wherein the reporter dye is different in the at least one further sensor. This allows for a sensor, or series of sensors, where a dye with very different properties is used. This can allow for more diversity to be brought to the sensor array.

The protein barrel may be immobilised on a substrate. The substrate may be, for example, a surface comprising a glass or plastics material. The protein barrel of any given sensor may be immobilised within the well of a multiwell plate. This would allow for washing and reuse of the protein barrel. The protein barrel of any given sensor may be immobilised on a flat surface, alongside neighbouring immobilised protein barrels from different sensors in the sensor array. This would allow for a single analyte to be readily applied across different sensors, without the protein barrels diffusing and interfering with each other. This would also allow for miniaturisation of the sensor array, allowing for a considerable number of sensors (i.e. perhaps at least 500 or at least 1000 sensors) to be present in a surface area of a small surface area (i.e. perhaps less than 5 or even less than 2 square centimetres). Such an array would provide a significant ability to distinguish between different analytes in a convenient and low-cost array. Such arrays are sometimes referred to as microchip arrays.

Techniques for immobilising protein barrels on a substrate are well-known (one example (Pai et al., 2012), discloses immobilisation of peptides in a microarray). Where the protein barrel comprises a number of self-assembled subunits, just one, multiple or all subunits may be individually immobilised. Typically, N- or C-terminal residues are used for immobilisation as this can lower the chance of disrupting the protein fold/3D structure. However, non-terminal residues may instead be used for linking the protein barrels to a substrate. For example, where an alpha protein barrel is used, an f position amino-acid residue could provide a suitable anchor point for immobilisation. Often, a flexible linker can be used between the protein barrel and the substrate to allow a certain degree of movement of the immobilised protein barrel.

The reporter dye can also be immobilised. The reporter dye can be immobilised to the substrate, by means of a linker that allows the reporter dye enough freedom of movement to enter and leave the protein barrel lumen. Alternatively, the reporter dye can be immobilised by linking to the protein barrel. Again, a linker should be used that allows the reporter dye enough freedom of movement to enter and leave the protein barrel. A different possibility is that the reporter dye is encapsulated within the lumen. In this possibility, the ends of the lumen would be blocked after the reporter dye has bound to the lumen. Immobilisation of the dye and barrel further allows for a sensor array that is reusable or can be used in-line, without needing to consider that either the protein barrel or dye may wash away.

The protein barrel and reporter dye can be in a dry state. By this, we mean that the complex of protein barrel and reporter dye have been dried. Drying can be carried out by techniques including air drying and lyophilisation. In the dry state, the sensor array can be stored and transported easily. Prior to use, the sensor array should be rehydrated. Rehydration can be achieved by adding an aqueous solution in advance of applying a test sample, or by adding an aqueous test sample.

These repeat sequences reflect repeat units of de novo alpha-helical barrels that form five-, six-, seven and eight-membered alpha-helical barrels.

While these repeat units represent the basic building block of an alpha helix, there may of course be point mutations such that not every unit is an identical repeat.

The analyte or complex mixture of analytes to be detected is in the sample obtained from a patient such as a human or animal, and is usually a liquid or in solution. It would also be advantageous to be able to analyse gaseous analytes such as breath. As an alternative to immobilisation on a solid substrate, the protein barrel can be immobilised in or on a hydrogel or 3-dimensional porous scaffold substrate. This has the advantage that the sensor array could be used to detect gaseous analytes, as these can be dissolved in the hydrogel and hence accessible to the barrel. In particular, the barrels can be loaded into hydrogels, or 3-dimensional porous scaffolds, either covalently or non-covalently. Polymers (such as poly(ethylene glycol), polydimethyl siloxane and polyacrylamide), polysaccharides (such as chitosan, alginate and agarose) and peptide hydrogels are examples of materials that could be used to form the hydrogels.

The invention also provides for a microarray chip comprising a sensor array according to the first aspect of the invention. Microarray chip technology is well known. The microarray chip can be 3D printed. The microarray chip can comprise the sensor array in a dry state, wherein an aqueous test sample is soaked onto the chip. The microarray chip may be analysable by a smartphone.

The sensor arrays of the invention provide significant amounts of data. It can be very difficult or even impossible for the human eye to detect the differences that distinguish between analytes, or complex mixtures of analytes as will likely be present in the samples. However, these differences are much more amenable to computational approaches. As such, step (d) may comprise the use of computational pattern recognition. Examples of computational pattern recognition used in the art include principal component analysis (PCA), linear discriminant analysis (LDA), hierarchical cluster analysis (HCA) and artificial neural networks (ANN).

### EXPERIMENTAL

### Synthesis of Protein Barrels

Alpha-helical barrels based on alpha helices with the following sequences (corresponding to the alpha-helical barrels referred to in FIG. 6) were synthesised.

| | Number | |
|---|---|---|
| | of | |
| | helices | |
| Peptide | in barrel | Sequence |
| CC-Hept-I17C | 7 | Ac-GEIAQALKEIAKALKE |
| | | CAWALKEIAQALKG-NH₂ |
| AFKEIA | 6 | Ac-GEIAQAFKEIAKAFKE |
| | | IAWAFKEIAQAFKG-NH₂ |
| AIKEIA | 8 | Ac-GEIAQAIKEIAKAIKE |
| | | IAWAIKEIAQAIKG-NH₂ |
| AIKEVA | 7 | Ac-GEVAQAIKEVAKAIKE |
| | | VAWAIKEVAQAIKG-NH₂ |
| AVKEVA | 6 | Ac-GEVAQAVKEVAKAVKE |
| | | VAWAVKEVAQAVKG-NH₂ |
| AVKEIA | 6 | Ac-GEIAQAVKEIAKAVKE |
| | | IAWAVKEIAQAVKG-NH₂ |
| AMKEIA | 7 | Ac-GEIAQAMKEIAKAMKE |
| | | IAWAMKEIAQAMKG-NH₂ |
| CC- | 5 | Ac-GQIEQILKQIEKILKQ |
| Pent_var(ILK | | IEWILKQIEQILKG-NH₂ |
| QIE) | | |
| CC-Hex | 6 | Ac-GELKAIAQELKAIAKE |
| (ELKAIA) | | LKAIAWELKAIAQG-NH₂ |
| CC-Hex2 | 6 | Ac-GEIAKSLKEIAKSLKE |
| (SLKEIA) | | IAWSLKEIAKSLKG-NH₂ |
| CC-Hept | 7 | Ac-GEIAQALREIAKALRE |
| (ALKEIA) | | IAWALREIAQALRG-NH₂ |
| CC-Hept-I24D | 7 | Ac-GEIAKALREIAKALRE |
| | | IAWALREDAKALRG-NH₂ |
| CC-Hept-I24E | 7 | Ac-GEIAKALREIAKALRE |
| | | IAWALREEAKALRG-NH₂ |
| CC-Hept-I24K | 7 | Ac-GEIAQALREIAKALRE |
| | | IAWALREKAQALRG-NH₂ |
| CC-Hept-I17K | 7 | Ac-GEIAQALREIAKALRE |
| | | KAWALREIAQALRG-NH₂ |

The peptide sequences were synthesised and characterized using techniques previously described (Thomson et al., 2014).

Fmoc amino acids, DMF and Cl-HOBt were purchased from AGTC Bioproducts (Hessle, UK). Rink amide ChemMatrix solid support was purchased from PCAS BioMatris Inc (Saint-Jean-sur-Richelieu, Canada). TMA-DPH and farnesyl pyrophosphate (FPP) were purchased from Sigma-Aldrich (Gillingham, UK). Farnesol was purchased from Alfa Aesar (Heysham, UK). All other chemicals were purchased from Fisher-Scientific (Loughborough, UK). Unless stated otherwise, biophysical measurements were performed in HEPES buffered saline (HBS; 25 mM HEPES, 100 mM NaCl, pH 7.0). Peptide concentration was determined by UV-Vis on a ThermoScientific (Hemel Hemstead, UK) Nanodrop 2000 spectrometer (ε₂₈₀=5690 cm⁻¹).

Standard Fmoc solid-phase peptide synthesis was performed on a CEM (Buckingham, UK) Liberty Blue automated peptide synthesis apparatus with inline UV monitoring. Activation was achieved with DIC/Cl-HOBt. Fmoc deprotection was performed with 20% v/v morpholine/DMF. All peptides were produced as the C-terminal amide on Rink amide ChemMatrix solid support and N-terminally acetylated upon addition of acetic anhydride (0.25 mL) and pyridine (0.3 mL) in DMF (5 mL) for 30 minutes at room temperature (rt). Peptides were cleaved from the solid support by addition of trifluoroacetic acid (9.5 mL), triisopropylsilane (0.25 mL) and water (0.25 mL) for 3 hours with shaking at rt. The cleavage solution was reduced to approximately 5 mL under a flow of nitrogen. Crude peptide was precipitated upon addition of diethyl ether (40 mL) and recovered via centrifugation. The resulting precipitant was dissolved in 1:1 acetonitrile and water (≈15 mL) and lyophilised to yield crude peptide as a while solid.

Peptides were purified by reverse phase HPLC on a Phenomenex (Macclesfield, UK) Luna C18 stationary phase column (150×10 mm, 5 μM particle size, 100 Å pore size). A 20-80% gradient of acetonitrile and water (with 0.1% TFA) was applied over 30 minutes. Fractions containing pure peptide were identified by analytical HPLC and MALDI-TOF MS, and were pooled and lyophilised.

### Binding of Dyes to Lumen

Initial experiments sought to demonstrate that reporter dyes would bind within the lumen of alpha-helical barrels. The dyes 1,6-diphenyl-1,3,5-hexatriene (DPH) and 6-propionyl-2-dimethylaminonaphthalene (prodan) were assayed against a number of alpha-helical barrels to determine their dissociation constants, K_{D}. DPH or prodan (1 μM) was incubated with varying concentrations of alpha-helical barrel (0.5-500 μM) for up to 2 hours, and the fluorescent signal measured at the corresponding emission wavelength.

| Peptide | DPH K_{D} (μM) | Prodan K_{D} (μM) |
|---|---|---|
| CC-Pent | 22.4 ± 4.3 | — |
| CC-Hex | 7.1 ± 1.3 | — |
| CC-Hex2 | 9.5 ± 1.1 | 39.2 ± 6.8 |
| CC-Hept | 8.9 ± 2.2 | 40.5 ± 4.0 |

It can be seen from the table above that DPH binds to all four alpha-helical barrels, while prodan did not bind to the alpha-helical barrels comprising CC-Pent or CC-Hex. Prodan did not bind as tightly to these alpha-helical barrels as DPH.

### Dye Displacement by Certain Analytes

After providing proof of concept that reporter dyes can bind within the lumen of alpha-helical barrels, the next step was to demonstrate that bound reporter dyes can be displaced by analytes. The four analytes below were selected based on having hydrophobic properties and being able to adopt an elongate configuration, as these were postulated to have the best chance of displacing a reporter dye.

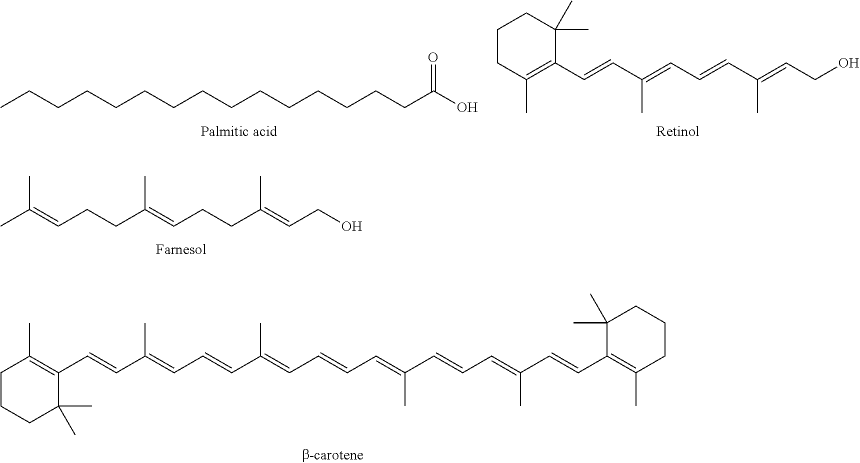

DPH was used as the reporter dye, and displacement of DPH was recorded using a standard competitive inhibition assay. In other words, the ability of an analyte to inhibit DPH binding was recorded by the inhibition constant Kᵢ. Alpha-helical barrels were incubated with DPH, or its cationic variant 1-(4-trimethylammoniumphenyl)-6-phenyl-1,3,5-hexatriene p-toluenesulfonate (TMA-DPH). Analyte was added (0.05-300 μM) and the fluorescence signal measured.

| | Palmitic acid | Retinol | Famesol | B-carotene |
|---|---|---|---|---|
| Peptide | K₁ (μM) | K₁ (μM) | K₁ (μM) | K₁ (μM) |
| CC-Pent | 1.1 ± 0.5 | 14.8 ± 4.1 | — | — |
| CC-Hex | 1.0 ± 0.3 | 6.4 ± 3.2 | 23.9 ± 2.4 | — |
| CC-Hex2 | 1.1 ± 0.3 | 4.6 ± 1.9 | 8.6 ± 1.3 | — |
| CC-Hept | 0.9 ± 0.3 | 4.0 ± 0.7 | 0.6 ± 0.2 | 12.1 ± 5.4 |

In all cases where competitive binding was observed, the inhibition constant was in the low micromolar range, similar to the dissociation constant of DPH indicating a similar strength of binding, and demonstrating that reporter dyes can be displaced by analytes.

Further evidence of analyte binding was provided by an x-ray crystal structure of farnesol bound within the lumen of the CC-Hex2 alpha-helical barrel. This is shown in FIGS. 5A and 5B. To obtain this crystal structure, a lyophilized sample of CC-Hex2 was resuspended in deionized water to a concentration of 5 mg ml⁻¹. Vapor-diffusion crystallization trials were set up at 19° C. using previously optimized conditions¹ (0.1 M Na HEPES, 4.3 M sodium chloride at pH 7.5) by mixing 1 μl of CC-Hex2 with 1 μl of reservoir solution. Diffraction-quality crystals were obtained in 4 days. A solution of farnesol (2 mM) was prepared in 40% v/v DMSO:H₂O and crystals were soaked for 1, 5, 20, 60 and 120 min. At each time point, the crystals were soaked in the reservoir solution containing 20% glycerol before freezing.

X-ray diffraction data were collected at the Diamond Light Source (Didcot, UK) on beamline 104-1 at a wavelength of 0.98 Å. Data were processed with MOSFLM (Battye et al., 2011) and AIMLESS (Evans and Murshudov, 2013), as implemented in the CCP4 suite (Winn et al., 2011). Due to high anisotropy in the diffraction data, the resultant mtz file was truncated to 2 Å in the b-axis using the Diffraction Anisotropy Server (Strong et al., 2006).

The crystal structure was solved by molecular replacement using a poly-alanine model of CC-Hex2 (PDB 4pn8). The structure was obtained after iterative rounds of model building with COOT (Emsley and Cowtan, 2004) and refinement with PHENIX refine (Afonine et al., 2012). Refinement was carried out with torsion-libration-screw (TLS) (Zucker, Champ and Merritt, 2010) and non-crystallographic symmetry (NCS) parameters. An Omit map was calculated from the final model after removal of the ligand and refinement in Phenix. Ligand structures and geometric restraints were calculated using Phenix eLBOW (Moriarty, Grosse-Kunstleve and Adams, 2009).

The final refined structure showed good stereochemistry, as analysed by MOLPROBITY (Chen et al., 2010) and Ramachandran plots indicated that no residues fell outside preferred regions of backbone conformational space.

### Differential Arrays

In a proof-of-principle experiment, 15 different alpha-helical barrel designs, as set out in FIG. 6, were arrayed in 96-well plates. The different alpha-helical barrels have a variety of sizes, with between 5 and 7 alpha helices. The different alpha-helical barrels have different charges, with some being neutral, some having negatively charged carboxylate groups in the lumen and some having positively charged ammonium groups in the lumen.

The reporter dye DPH was added to each well and allowed to bind within the lumens of each alpha-helical barrel. Seven different small and large molecules were then subjected to the sensor assay. The molecules and the optical signal of each sensor in each sensor assay is shown in FIG. 7. This Figure shows a unique binding signature for each of the molecules.

It is important to realise the significance of the molecules screened. Cholesterol and nervonic acid are largely hydrophobic molecules that might be expected to bind readily within the lumen of an alpha-helical barrel. Furthermore, both can act as biomarkers, cholesterol for cardiovascular disease and nervonic acid for psychoses.

Dimethylarginine and N-acetyl-L-aspartic acid are highly polar amino acids, bearing multiple charges. It might be expected that these molecules would have little effect on an alpha-helical barrel with an uncharged and hydrophobic lumen, however, a displacement pattern is seen even across such alpha-helical barrels.

Hexamethyltetramine is an explosives precursor and again produces a distinct displacement pattern. Triisopropylphosphate is a sterically bulky nerve agent analogue.

A significant result was the sensor array pattern produced by insulin. Insulin is a peptide that should not be able to fit within the lumen of the alpha-helical barrels used in the assay. However, a unique reporter dye displacement pattern was still produced. This provides evidence that even when analytes interact with the outer surface of an alpha-helical barrel, reporter dye displacement can occur.

High reproducibility was observed in repeat assays, as can be seen for the replicate data presented in FIG. 9.

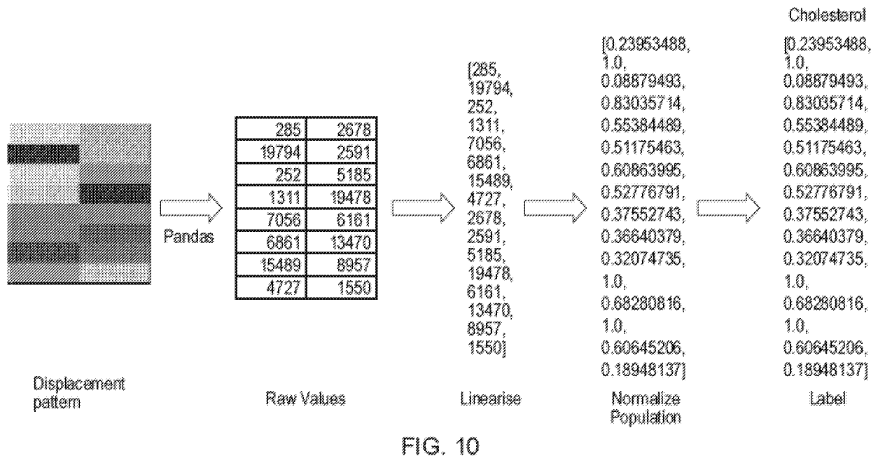

FIG. 10 shows a workflow for applying computational pattern recognition to the sensor array results. The raw data is normalised, before looking for patterns that uniquely identify the analyte. By applying machine learning to the sensor array patterns for each molecule, the predictive power showed greater than 95% correct predictions.

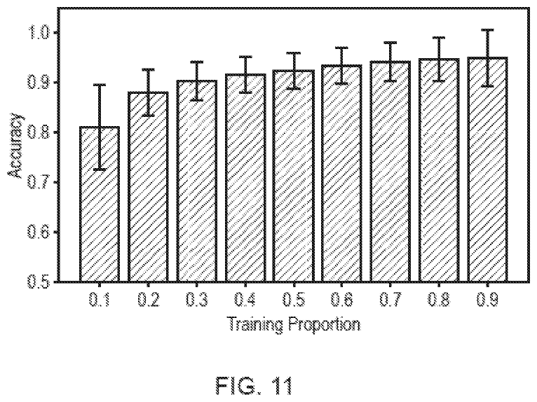

FIG. 11 shows how the prediction of analytes from naïve (unseen) data improves as the proportion of the data from known training sets is increased. In this case, by using random selection of just ≈30% of the 150 datasets of array signatures recorded for each of the known compounds, >90% of the predictions from the non-training-sets data are correct.

### Analysing Complex Mixtures

A selection of teas was analysed as a test bed for the analysis of complex mixtures. A total of 9 different boxes of tea bags where purchased from local supermarkets. This comprised three black teas (PG Tips, Yorkshire Tea, and Pukka English Breakfast), three Earl Grey Teas (Twinings The Earl Grey, Pukka Gorgeous Earl Grey, and Clipper Organic Earl Grey), and three Green Teas (Clipper Organic Green Tea, Twinings Pure Green Tea, and Tetley Pure Green Tea).

Teas were brewed in the laboratory as follows: Firstly, when applicable, strings and labels were removed from tea bags. Next, deionised water was boiled in a newly purchased kettle free of limescale. A single tea bag was placed in a 500 mL Schott bottle with a 50 mm stirrer bar before 250 mL of deionised water was added, and the tea allowed to brew for 5 min with stirring (100 rpm). After this time, 1 mL of the tea solution was removed, and diluted 1:10 with deionised water and the solution snap frozen in liquid nitrogen and then stored at −80° C. Fresh tea samples were prepared for each experimental replicate using an identical protocol.

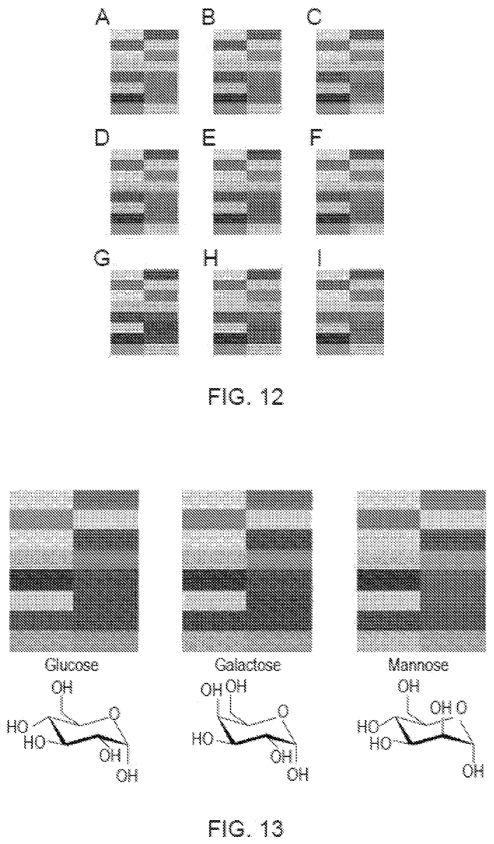

Using a suite of 15 barrel-forming peptide, plus a non-peptide containing control, tea was analysed by observing DPH displacement to yield fingerprints as depicted in FIG. 12. FIG. 12 shows the DPH displacement fingerprints produced by selected tea samples as follows: Panel A PGTIPS; Panel B Pukka English Breakfast; Panel C Yorkshire Tea; Panel D Clipper Organic Earl Grey; Panel E Pukka Gorgeous Earl Grey; Panel F Twinings The Earl Grey; Panel G Clipper Organic Green Tea; Panel H Tetley Pure Green Tea; and Panel I Twinings Pure Green Tea.

Implementing machine leaning techniques, tea could be successful classified by class (i.e. Black, Earl Grey or Green Tea) with 82.3% accuracy and by specific type with 90.0% accuracy.

### Analysing Epimers.

Glucose, galactose and mannose were analysed in an array of 15 peptides and a DPH control. These three sugars are epimers in that they differ by configuration and a single stereo-centre. Solutions of each of the three were prepared at 10 mM concentration ion water before being analysed at 1 mM final concentration in the barrel array in which DPH displacement was measured. Each sugar was examined using 24 replicates of each barrel, in each of two 384-well plates on two separate days (i.e. 4 plates for each sugar). The peptide array was able to distinguish between these 3 very similar molecules as shown by FIG. 13 which depicts the DPH displacement fingerprints for glucose, galactose and mannose across the top, and across the bottom the structure of each of the epimers.

### Non-Natural Amino Acids.

To demonstrate the use of non-natural amino acids, 3 non-proteinogenic peptides were incorporated into the array of 15 barrels and a DPH control by replacing 3 proteinogenic peptides.

Proteinogenic array

| | |
|---|---|
| DPH control | CC-Pent_var (ILKQIE) |
| CC-Hept-I17C | CC-Hex (ELKAIA) |
| AFKEIA | CC-Hex2 (SLKEIA) |
| AIKEIA | CC-Hept (ALKEIA) |
| AIKEVA | CC-Hept-I24D |
| AVKEVA | CC-Hept-I24E |
| AVKEIA | CC-Hept-I24K |
| AMKEIA | CC-Hept-I17K |

Non-proteinogenic array

| | |
|---|---|
| DPH control | CC-Pent_var (ILKQIE) |
| CC-Hept-I17C | CC-Hex (ELKAIA) |
| AFKEIA | CC-Hex2 (SLKEIA) |
| AIKEIA | CC- Hept (ALKEIA) |
| AIKEVA | CC-Hept-124D |
| AVKEVA | CC-Hex-L24E |
| AVKEIA | CC-Hept-L28NIE |
| AMKEIA | CC-Hept-dL (AdLKEIA) |

As can be seen, peptides in the standard proteinogenic array are shown on the left and the non-proteinogenic array on the right incorporates 3 peptide sequences with unnatural amino acids. Nle=Norleucine, dL=Dehydroleucine.

CCHept-L28Nle:
Ac-GEIAQALKEIAKALKEIAWALKEIAQANleKG-NH2
CCHept-dL:
Ac-GEIAQAdLKEIAKAdLKEIAWAdLKEIAQAdLKG-NH2
CCHex-L24Nle:
(SEQ ID NO: 46)
Ac-GELKAIAQELKAIAKELKAIAWENleKAIAQG-NH2

Cholesterol was analysed at 1 μM and the DPH displacement fingerprints analysed.

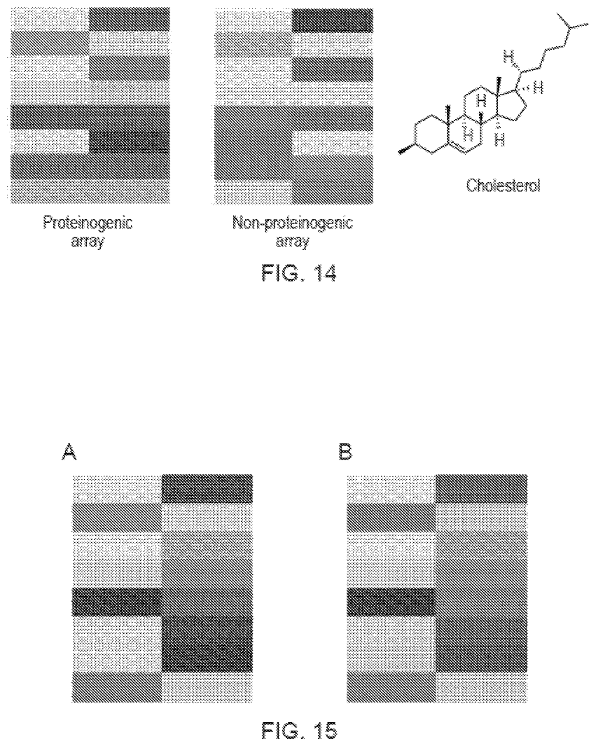

As can be seen in FIG. 14, a clear difference is observed when the proteinogenic (on the left) and non-proteinogenic (on the right) fingerprints are compared.

### D Amino Acid Peptides.

To demonstrate the use of D-amino acids in the barrel array, an analogue of peptide ALKEVA comprising entirely D-Amino acids was prepared (i.e. peptide d-(AVKEVA), below)

d-(AVKEVA):
(SEQ ID NO: 45)
Ac-GevaqavkevakavkevawavkevaqakvG-NH₂

This peptide, which possesses the opposite chirality to peptide ALKEVA at each chiral centre, was substituted into a 15 peptide barrel array (as listed in Example 1) in place of peptide AVKEIA. Using this modified array, two small molecules were analysed for DPH displacement: N-Acetyl-L-aspartic acid and NG,NG-Dimethylarginine. Solutions of each molecule were prepared at 10 μM in water before being examined at 1 μM concentration with 24 replicates in each of three 384-well plates. FIG. 15 shows the DPH displacement signatures for each of these two molecules. In particular FIG. 15 shows DPH displacement fingerprints for N-Acetyl-L-aspartic acid (Panel A) and NG,NG-Dimethylarginine (Panel B) using a peptide barrel array including one all D-amino acid peptide (d-(AVKEVA)) which is represented by the block depicted on the left, second from bottom in each fingerprint. From these data, machine learning techniques were implemented and the two molecules distinguished with 95.5% accuracy.

### Example

This example demonstrates that the sensor array technology can distinguish between the varying secretome produced by non-cancerous cells, cells derived from primary tumours, and those from secondary tumours.

A total of 10 cell lines were employed, all of mouse origin: 3 Non-cancerous (NMuMg, HC11, and EpH4), 3 of primary mammary tumour origin (Yej, 113, and 734), and 4 of metastasised mammary tumour origin (Yej-M1, Yej-M2, 113-M1, and 113-M2). Table 1 summarises the cell lines used in the current study. It should also be noted that the cell lines Yej, Yej-M1, and Yej-M2 are iso-genetic—that is to say that the lines Yej-M1 and Yej-M2 are each derived from secondary tumours produced from the fat pad transplant and growth of a Yej derived tumour in a recipient mouse. In a similar fashion, the lines 113, 113-M1 and 113-M2 are also isogenetic, although in this instance 113-M1 and 113-M2 are derived from lung metastasis following tail vein injection of the 113 primary cell line.

TABLE 1
Cell lines used in the present study.

| Non- | Primary | Metastasised |
|---|---|---|
| Cancerous | Tumour | Tumour |
| NMuMg | Yej | Yej -M1 |
| HC11 | 113 | Yej -M2 |
| EpH4 | 724 | 113-M1 |
| | | 113-M2 |

### Preparing the Samples—Cell Lines and Conditioned Media

NMuMg, EpH4 and HC11 cells are epithelial cells derived from normal glandular mouse tissues (commercially available). Mammary tumour cell lines were made at the CRUK Beatson Institute, Glasgow, from spontaneous tumours arising in the MMTV-PyMT mouse model of breast cancer. In this model, the PyMT oncogene is expressed under control the control of the mammary gland specific MMTV-LTR promoter, resulting in well characterised disease progression that recapitulates the key events occurring in human metastatic breast cancer. Tumours measuring a maximum size of 9 mm×9 mm were excised from the mouse, processed to a pate texture using a tissue chopper, and then digested in collagenase/hyaluronidase (15000 U Collagenase/5000 U hyaluronidase) for 1-2 hours at 37° C. with gentle shaking. Samples were then centrifuged for 1 minute at 15 g, and the supernatant collected. Supernatant was then centrifuged at 100 g for 3 minutes, and the consequent supernatant then centrifuged at 400 g for 10 minutes. The supernatant was then discarded, the cell pellet resuspended in full growth media, and then centrifuged at 800 r.p.m. for 3 minutes to wash the cells. This wash step was repeated a further two times, and then cells were resuspended in full growth media and incubated and maintained at 37° C./5% CO₂ for passaging.

Metastatic variants of the mammary tumour cell lines were made using a fat pad transplantation model. In short, 0.5 million tumour cells were injected into the fourth mammary fat pad of recipient mice, and tumours allowed to grow until 9 mm×9 mm measurable size. Tumours were then surgically removed and the recipients allowed to recover, with weight and general health monitored over time. Recipients were culled upon signs of metastatic disease, including cachexia, weight loss and difficulty breathing. Lungs were harvested and processed as described above, with metastatic tumour cell lines consequently being isolated from the lungs of recipients that had succumbed to lung metastasis.

Normal mouse mammary epithelial cells, primary mammary tumour cell lines, and metastatic variants of the primary tumour cell lines, were maintained in DMEM supplemented with 10% FBS, 2 mM L-Glutamine, 10 ug/mL Insulin, 20 ng/mL EGF and 100 U/L Penicillin-Streptomycin at 37° C./5% CO₂. Cells were plated at a density of 2×10⁶ cells per 10 cm dish in 10 mL total volume, and incubated at 37° C./5% CO₂ for 24 hours. Conditioned media was then collected and subjected to the following differential centrifugation protocol: 300 g for 10 minutes, 2000 g for 10 minutes, and 10000 g for 30 minutes, with all centrifugation steps conducted at 4° C. The resulting cell culture supernatant was then snap frozen and stored at −80° C. before use in the sensor array. Cell counts were also performed at the point of conditioned media collection in order to enable normalisation to final cell number. For each cell line, conditioned media was collected across three separate days to give n=3. Thus, with 10 different cell lines (3 non-cancer, 3 primary, and 4 metastatic) used, and conditioned media collected 3 times we examined 30 different batches of media.

Contact with Sensor Array

Before analysis in the sensor array, frozen conditioned media samples were defrosted and diluted relative to the cell count measured at the time media was collected. These cell counts ranged from 1.67×10⁵ cell/mL to 6.84×10⁵ cell/mL. Final concentration of media in sample ranged from 2.0% (for the conditioned media with the lowest cell count) to 0.49% (for the media with the highest cell count).

The analysis of conditioned media samples was performed as outlined in above, using the sensor array described at the beginning of the Experimental section above. Briefly, a set of 15 barrel-forming coiled coil peptides (plus a single no-peptide control) were arrayed (at 10 μM in HEPES buffered saline) with diphenylhexatriene (DPH; 1 μM) on a 384 well plate (i.e. each peptide plus control was deposited in 24 replicates per plate). Next, a given conditioned media analyte was added across columns 1-5, 8-14, and 17-24 of the plate. An equal volume of water was added to columns 6, 7, 15, & 16 to serve as a control. After 1 h, DPH fluorescence was measured (350/450 nm, excitation/emission) and, for each analyte-containing well, normalised to control well value obtained for that given barrel peptide. Each conditioned media sample was assayed on 4 separate 384 well plates, across 4 different days to give n=4.

### Results—Generation of Fingerprints.

For each sample of conditioned media, normalised DPH fluorescence data from each barrel-forming peptide was averaged across each of the four plates. As described above, colour graduation can be used to represent this average fluorescence from each of the 15 barrel (plus −ve control) as a 16 cell fingerprint.

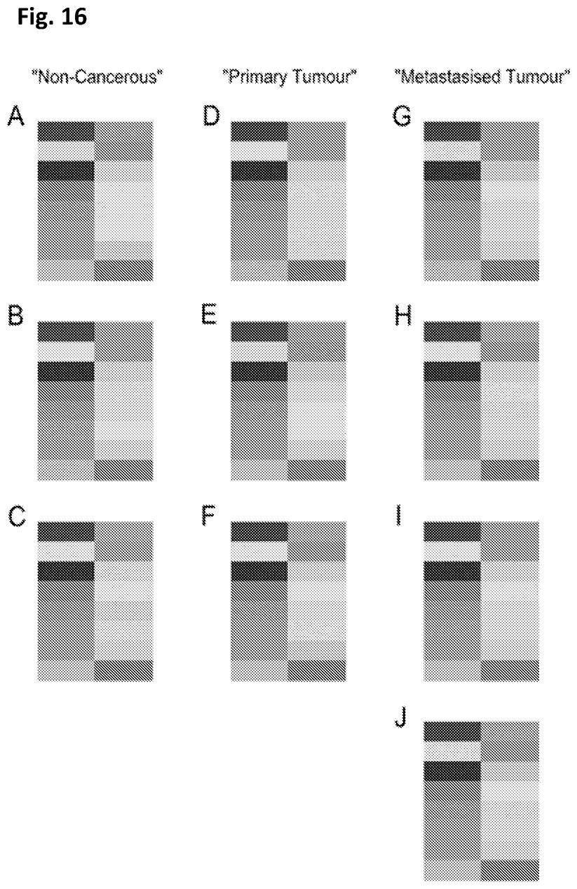

FIG. 16 shows the fingerprints generated for all 10 conditioned media samples. This includes media collected from cells of “non-cancerous”, “primary tumour”, and “metastasised tumour” origins as indicated. Each fingerprint conditioned media is labelled as follows. A: NMuMg; B: HC11; C:EpH4; D: Yej; E: 113; F: 724; G: Yej-M1; H: Yej-M2; I: 113-M1; J: 113-M2.

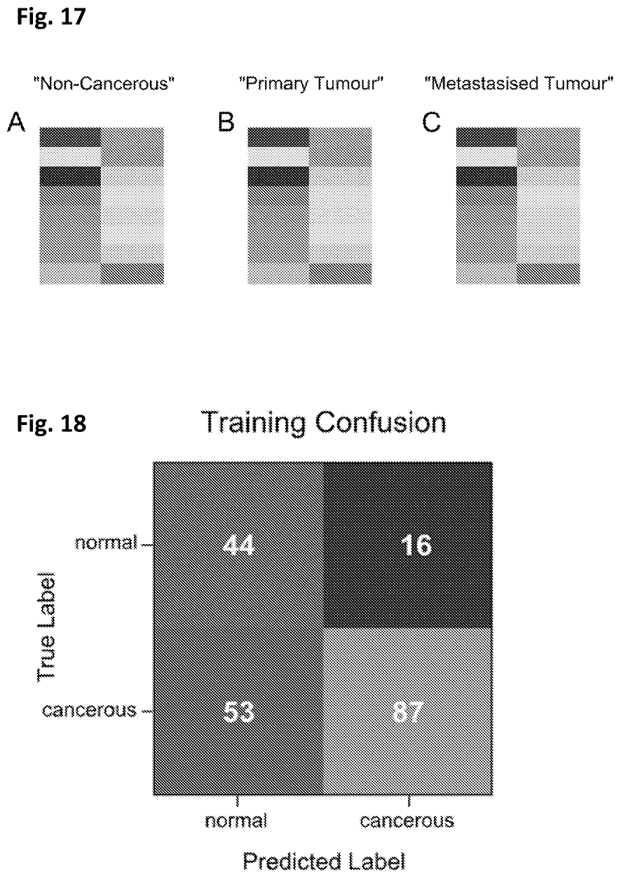

FIG. 17 shows the fingerprints generated for combined “non-cancerous” (panel A), “primary tumour” (B) and metastasised tumour (C) derived conditioned media.

### Results—Machine Learning Algorithms

Using machine learning techniques, we were able to successfully categorise the cells as being from cancerous or non-cancerous origin with 65.5% accuracy. Taking this analysis a step further, attempting a 3-way classification for non-cancer vs primary cancer vs. metastasised cancer returned an accuracy of 47.5% (baseline “guessing” would return only 33%). And finally, focussing exclusively on primary and metastatic tumour-derived samples, returned an accuracy of 67.1% in being able to distinguish between the two. It is expected that with a larger dataset and further use of pattern recognition and artificial intelligence the accuracy will greatly improve going forward. Confusion matrices for each of these analysis are shown in FIGS. 18, 19 and 20.

FIG. 18 is the confusion matrix for 2-way prediction of healthy, non-cancerous cells, and cells originating tumours.

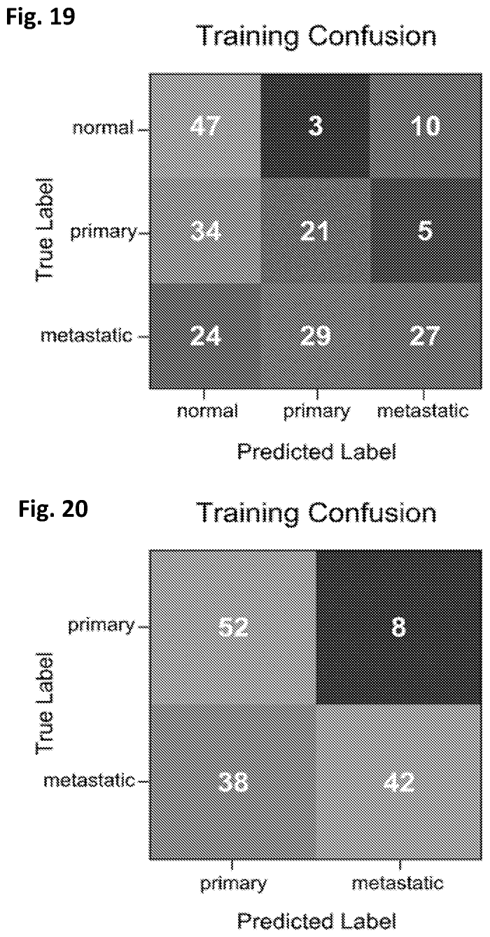

FIG. 19 is the confusion matrix for 3-way prediction of healthy, non-cancerous cells, and cells originating from primary tumours and metastatic tumours.

FIG. 20 is the confusion matrix for 2-way prediction of cells originating from primary tumours and metastatic tumours.

### Interrogating the Sensor Fingerprint In Vitro

Fractionation approaches can be used to interrogate the secretome of the primary tumour cells, and their metastatic variants, in order to inform which components are responsible for distinguishing the fingerprint of a non-cancer versus cancerous sample, and primary versus metastatic samples. A variety of approaches can be used to understand whether these distinguishing features are constituents of either exosomes, the water soluble compartment, or the lipid soluble compartment of the samples.

With respect the exosome content, centrifugation of the samples at 100,000 g at 4° C. for 70 minutes can be used to isolate the exosomes from the conditioned media of the described cell lines, with consequent use of the Sensor array to fingerprint exosome depleted samples, and enable us to understand whether or not the exosomes are a distinguishing factor in this analysis.

To the same end, we can also deplete secreted proteins from such samples to understand whether or not the secreted proteome is also a contributing factor. In this case, conditioned media are centrifuged at 300 g for 10 minutes at 4° C., supernatant collected and centrifuged at 2000 g for 10 minutes at 4° C., and supernatant then collected and centrifuged at 10,000 g for 30 minutes at 4° C. Consequent supernatant is then acidified to pH5 with 10% TFA and 10 uL Strataclean (hydroxylated silica) beads added per 1 mL of media. The media/bead slurry is then vortexed for 1 minute and incubated overnight on a rotor wheel at 4° C. The beads are then collected by brief centrifugation, with secreted proteins then being bound to the beads, therefore leaving then conditioned media depleted of proteins and available for fingerprinting for the sensor array according to the invention.

We also have the ability to isolate metabolites and lipids from such samples, and therefore to implement these approaches in this analysis. With regards to the metabolomics, metabolites are extracted in a polar solvent (50% methanol, 30% acetonitrile, 20% water) and centrifuged to precipitate and remove any proteins present. These extracts can then be applied to the sensor array to obtain a fingerprint for the non-cancer, primary and metastatic samples, whilst in parallel we use HILIC liquid chromatography (LC) coupled with high resolution Orbitrap mass spectrometry (Thermo Scientific) to profile the polar metabolites in these samples in an untargeted fashion. In reference to the lipid component of the secretome, lipids can be extracted in a two-step procedure by the Folch method. The biological samples are treated with a mixture of chloroform and methanol, forming bi-phasic layers, and the chloroform layer are then subsequently evaporated and reconstituted in a compatible organic solvent. We again have the ability to test the lipid extracts on the sensor array, whilst also profiling the contents of those samples in parallel to characterise any differences in the samples. In short, lipids are separated using reversed-phase (RP) liquid chromatography using C18 columns as well as mobile phase modifiers. We use two chromatographic methods to separate lipids:
The general lipidomics method separates lipid species using a gradient of solvents such as water, acetonitrile, and isopropanol, as well as ammonium formate as modifier. This method allows the identification of more than 20 lipid classes, including the triacylglycerol (TG), phosphatidyl ethanolamine (PE), phosphatidyl choline (PC), and ceramide (Cer) families.
The polar lipidomics method uses only water and methanol in the chromatographic gradient, and we use ammonia as modifier. This is useful when the intention is to analyse polar lipids that are not detected in the general method, such as lysophosphatidic acid (LPA).

We can then use high resolution Orbitrap mass spectrometry in separate polarity modes and data-dependent fragmentation acquisition (ddMS2), with lipid identification being dependent on both accurate mass and fragmentation patterns. Both of these methods will enable us to extract, fingerprint and define the metabolite and lipid composition of the samples.

### Interrogating the Sensor Fingerprint In Vivo

The above approaches can also be applied to samples derived from our mouse models of cancer. We can test the sensor array's ability to distinguish between the serum of mice derived from different genetic backgrounds. We can apply the principles described above to whole and fractionated sera from mouse models of cancer, and to sera from healthy volunteers and cancer patients.

### REFERENCES

Adams, M. M.; Anslyn, E. V. Journal of the American Chemical Society 2009, 131, 17068-17069
Afonine, P. V.; Grosse-Kunstleve, R. W.; Echols, N.; Headd, J. J.; Moriarty, N. W.; Mustyakimov, M.; Terwilliger, T. C.; Urzhumtsev, A.; Zwart, P. H.; Adams, P. D. Acta Crystallographica Section D-Biological Crystallography 2012, 68, 352.
Battye, T. G. G.; Kontogiannis, L.; Johnson, O.; Powell, H. R.; Leslie, A. G. W. Acta Crystallographica Section D-Biological Crystallography 2011, 67, 271.
Collie, G. W.; Pulka-Ziach, K.; Lombardo, C. M.; Fremaux, J.; Rosu, F.; Decossas, M.; Mauran, L.; Lambert, O.; Gabelica, V.; Mackereth, C. D.; Guichard, G. Nature Chemistry 2015, 7, 871-878.
Chen, V. B.; Arendall, W. B.; Headd, J. J.; Keedy, D. A.; Immormino, R. M.; Kapral, G. J.; Murray, L. W.; Richardson, J. S.; Richardson, D. C. Acta Crystallographica Section D-Biological Crystallography 2010, 66, 12.
Diehl, K. L.; Ivy, M. A.; Rabidoux, S.; Petry, S. M.; Müller, G.; Anslyn, E. V. Proceedings of the National Academy of Sciences of the USA 2015, 112, E3977-E3986.
Donadelli M. The cancer secretome and secreted biomarkers. Semin Cell Dev Biol. 2018:78:1-2.
Emsley, P.; Cowtan, K. Act. Cryst. D 2004, 60, 2126.
Evans, P. R.; Murshudov, G. N. Acta Crystallographica Section D-Biological Crystallography 2013, 69, 1204.
Fletcher, J. M. et al. ACS Synthetic Biology 2012, 1, 240-250.
Ghanem, E.; Afsah, S.; Fallah, P. N.; Lawrence, A.; LeBovidge, E.; Raghunathan, S.; Rago, D.; Ramirez, M. A.; Telles, M.; Winkler, M.; Schumm, B.; Makhnejia, K.; Portillo, D.; Vidal, R. C.; Hall, A.; Yeh, D.; Judkins, H.; Ataide da Silva, A.; Franco, D. W.; Anslyn, E. V. ACS Sensors 2017, 2, 641-647.
Hanahan D, Weinberg R A. The hallmarks of cancer. Cell. 2000; 100(1):57-70.
Hanahan D, Weinberg R A. The hallmarks of cancer: the next generation. Cell. 2011:144(5):646-74.
Ivy, M. A.; Gallagher, L. T.; Ellington, A. D.; Anslyn, E. V. Chemical Science 2012, 3, 1717-2176.
Koronakis, V.; Sharff, A.; Koronakis, E.; Luisi, B.; Hughes, C. Nature 2000, 405, 914-919.
Kubarych, C. J.; Adams, M. M.; Anslyn E. V. Organic Letters 2010, 12, 4780-4783.
Liotta L A, Ferrari M, Petricoin E. Clinical proteomics: written in blood. Nature. 2003; 425:905 Tjalsma H, Bolhuis A, Jongbloed J D, Bron S, van Dijl J M. Signal Peptide-Dependent Protein Transport in Bacillus subtilis: a Genome-Based Survey of the Secretome. Microbiol Mol Biol Rev. 2000; 64:515-547
Lombardo, C. M.; Collie, G. W.; Pulka-Ziach, K.; Rosu, F.; Gabelica, V.; Mackereth, C. D.; Guichard, G. Journal of the American Chemical Society 2016, 138, 10522-10530.
Malashkevich, V. N.; Kammerer, R. A.; Efimov, V. P.; Schulthess, T.; Engel, J. Science 1996, 274, 761-765.
Meusch, D. et al. Nature 2014, 508, 61-65.
Moriarty, N. W.; Grosse-Kunstleve, R. W.; Adams, P. D. Acta Crystallographica Section D-Biological Crystallography 2009, 65, 1074.
Novo D, Heath N, Mitchell L, Caligiuri G, MacFarlane A, Reijmer D, Charlton L, Knight J, Calka M, McGhee E, Dornier E, Sumpton D, Mason S, Echard A, Klinkert K, Secklehner J, Kruiswijk F, Vousden K, Macpherson I R, Blyth K, Bailey P, Yin H, Carlin L, Morton J, Zanivan S, Norman J. Nat Commun. 2018: 9: 5069.
Pai, J.; Yoon, T.; Kim, N. D.; Lee, I. S.; Yu, J.; Shin, I. Journal of the American Chemical Society 2012, 134, 19287-19296.
Rhys, G.; Wood, C.; Lang, E.; Mulholland, A.; Brady, R.; Thomson, A.; Woolfson, D. Nature Communications 2018, 9; 4132.
Strong, M.; Sawaya, M. R.; Wang, S. S.; Phillips, M.; Cascio, D.; Eisenberg, D. Proceedings of the National Academy of Sciences of the United States of America 2006, 103, 8060.
Sun, L. et al. Nature 2014, 505, 432-435.
Thomas, F.; Dawson, W.; Lang, E.; Burton, A.; Bartlett, G.; Rhys, G.; Mulholland, A.; Woolfson, D. ACS Synth. Biol. 2018, 7, 1808-1816.
Thomson, A. R.; Wood, C. W.; Burton, A. J.; Bartlett, G. J.; Sessions, R. B.; Brady, R. L.; Woolfson, D. N. Science 2014, 346, 485-488.
Umali, A. P.; Anslyn, E. V. Curr. Op. Chem. Biol 2010, 14, 685-692.
Umali, A. P.; Ghanem, E.; Hopfer, H.; Hussain, A.; Kao, Y.; Zabanal, L. G.; Wilkins, B. J.; Hobza, C.; Quach, D. K.; Fredell, M.; Heymann, H.; Anslyn, E. V. Tetrahedron 2015, 71, 3095-3099.
Winn, M. D.; Ballard, C. C.; Cowtan, K. D.; Dodson, E. J.; Emsley, P.; Evans, P. R.; Keegan, R. M.; Krissinel, E. B.; Leslie, A. G. W.; McCoy, A.; McNicholas, S. J.; Murshudov, G. N.; Pannu, N. S.; Potterton, E. A.; Powell, H. R.; Read, R. J.; Vagin, A.; Wilson, K. S. Acta Crystallographica Section D-Biological Crystallography 2011, 67, 235.
You, L.; Zha, D.; Anslyn, E. V. Chemical Reviews 2015, 115, 7840-7892.
Zaccai, N. R.; Chi, B.; Thomson, A. R.; Boyle, A. L.; Bartlett, G. J.; Bruning, M.; Linden, N.; Sessions, R. B.; Booth, P. J.; Brady, R. L.; Woolfson, D. N. Nature Chemical Biology 2011, 7, 935-941.
Zucker, F.; Champ, P. C.; Merritt, E. A. Acta Crystallographica Section D-Biological Crystallography 2010, 66, 889.

1. A method of diagnosing, staging or monitoring cancer, the method comprising the steps of:
(a) providing a sensor array comprising at least two sensors, wherein each sensor comprises a protein barrel that comprises five or more alpha helices arranged as an alpha-helical barrel, and a reporter dye, wherein the protein barrel defines a lumen, the reporter dye is bound to the lumen reversibly; and wherein the protein barrel is different in structure in the at least two sensors;
(b) contacting the sensor array with a sample obtained from a patient; and then
(c) comparing the sensor array to a predetermined standard.

2. The method according to claim 1, wherein the sample is liquid in which tumour or tissue cells from the patient have been cultured.

3. The method according to claim 1, wherein the sample is or is obtained from whole blood, a cell scraping, a biopsy tissue, bone marrow, plasma, serum, cerebrospinal fluid, saliva, semen, sputum, urine or stool.

4. The method according to claim 1, wherein the cancer is breast cancer.

5. The method according to claim 1, wherein the cancer is metastatic breast cancer in the lung.

6. The method according to claim 1, wherein each alpha helix independently comprises a sequence having a repeat unit with sequence abcdefg, wherein 50% or more of the a and d positions are hydrophobic amino acids and wherein 50% or more of the b, c, e, f and g positions are polar amino acids.

7. The method according to claim 6, wherein the repeat unit with sequence abcdefg is selected from the list consisting of: LQKIEfI, LKAIAfE, LKEIAfS, IKEIAfS, LKEIAfA, FKEIAfA, IKEIAfA, IKEVAfA, VKEVAfA, VKEIAfA, MKEIAfA, LKQIEfI, LKEVAfA, VKELAfA, IKELSfA, IKELAfS, LKELAfS, FKEIAfA, LKQIEfI and LKELAfA; wherein f may vary between repeat units.

8. The method according to claim 1, wherein each alpha helix comprises at least three repeat units.

9. The method according to claim 1, wherein the protein barrel comprises a non-natural amino acid.

10. The method according to claim 9, wherein the non-natural amino acid is an amino acid that has been modified by chemically linking a protein substrate.

11. The method according to claim 10, wherein the protein substrate comprises an enzyme substrate, receptor substrate and/or antibody substrate.

12. The method according to claim 1, wherein the protein barrel comprises a single and continuous amino acid backbone.

13. A sensor array according to claim 1, wherein the protein barrel is immobilised on a substrate, preferably wherein the substrate is a solid substrate or is a hydrogel.

14. The method according to claim 1, wherein the protein barrel and reporter dye are in a dry state.

15. The method according to claim 1, wherein the reporter dye provides an optical signal when bound to the lumen.

16. The method according to claim 1, wherein the reporter dye is a compound according to Formula I: 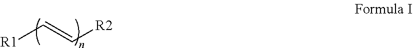
wherein n is 3 or more, preferably n is 3, 4 or 5, more preferably n is 3; and
R1 and R2 are independently selected from aryl or heteroaryl, preferably aryl, more preferably phenyl.

17. The method according to claim 1, comprising at least 10 sensors, preferably at least 50 sensors, more preferably at least 100 sensors, yet more preferably at least 300 sensors, wherein the protein barrel is different in each of the at least 10, 50, 100 or 300 sensors respectively.

18. The method according to claim 1, comprising at least one further sensor, wherein the reporter dye is different in the at least one further sensor.

19. The method according to claim 1, wherein the sensor array is incorporated into a microarray chip.

20. A method according to claim 1, wherein step (d) comprises computational pattern recognition.

21. Use of a sensor array comprising at least two sensors, wherein each sensor comprises a protein barrel that comprises five or more alpha helices arranged as an alpha-helical barrel, and a reporter dye, wherein the protein barrel defines a lumen, the reporter dye is bound to the lumen reversibly; and wherein the protein barrel is different in structure in the at least two sensors, to diagnose, stage or monitor cancer.


SEQUENCE LISTING (48 sequences)

SEQ ID NO: 1 - protein, 7 aa, Artificial Sequence
  Synthetic peptide
  misc_feature (6)..(6): Xaa can be any naturally occurring amino acid
LQKIEXI

SEQ ID NO: 2 - protein, 7 aa, Artificial Sequence
  Synthetic Peptide
  misc_feature (6)..(6): Xaa can be any naturally occurring amino acid
LKAIAXE

SEQ ID NO: 3 - protein, 7 aa, Artificial Sequence
  Synthetic Peptide
  misc_feature (6)..(6): Xaa can be any naturally occurring amino acid
LKEIAXS

SEQ ID NO: 4 - protein, 7 aa, Artificial Sequence
  Synthetic Peptide
  misc_feature (6)..(6): Xaa can be any naturally occurring amino acid
IKEIAXS

SEQ ID NO: 5 - protein, 7 aa, Artificial Sequence
  Synthetic Peptide
  misc_feature (6)..(6): Xaa can be any naturally occurring amino acid
LKEIAXA

SEQ ID NO: 6 - protein, 7 aa, Artificial Sequence
  Synthetic Peptide
  MISC_FEATURE (6)..(6): Xaa can be any naturally occuring amino acid
FKEIAXA

SEQ ID NO: 7 - protein, 7 aa, Artificial Sequence
  Synthetic Peptide
  misc_feature (6)..(6): Xaa can be any naturally occurring amino acid
IKEIAXA

SEQ ID NO: 8 - protein, 7 aa, Artificial Sequence
  Synthetic Peptide
  misc_feature (6)..(6): Xaa can be any naturally occurring amino acid
IKEVAXA

SEQ ID NO: 9 - protein, 7 aa, Artificial Sequence
  Synthetic Peptide
  misc_feature (6)..(6): Xaa can be any naturally occurring amino acid
VKEVAXA

SEQ ID NO: 10 - protein, 7 aa, Artificial Sequence
  Synthetic Peptide
  misc_feature (6)..(6): Xaa can be any naturally occurring amino acid
VKEIAXA

SEQ ID NO: 11 - protein, 7 aa, Artificial Sequence
  Synthetic Peptide
  misc_feature (6)..(6): Xaa can be any naturally occurring amino acid
MKEIAXA

SEQ ID NO: 12 - protein, 7 aa, Artificial Sequence
  Synthetic Peptide
  misc_feature (6)..(6): Xaa can be any naturally occurring amino acid
LKQIEXI

SEQ ID NO: 13 - protein, 7 aa, Artificial Sequence
  Synthetic Peptide
  misc_feature (6)..(6): Xaa can be any naturally occurring amino acid
LKEVAXA

SEQ ID NO: 14 - protein, 7 aa, Artificial Sequence
  Synthetic Peptide
  misc_feature (6)..(6): Xaa can be any naturally occurring amino acid
VKELAXA

SEQ ID NO: 15 - protein, 7 aa, Artificial Sequence
  Synthetic Peptide
  misc_feature (6)..(6): Xaa can be any naturally occurring amino acid
IKELSXA

SEQ ID NO: 16 - protein, 7 aa, Artificial Sequence
  Synthetic Peptide
  misc_feature (6)..(6): Xaa can be any naturally occurring amino acid
IKELAXS

SEQ ID NO: 17 - protein, 7 aa, Artificial Sequence
  Synthetic Peptide
  misc_feature (6)..(6): Xaa can be any naturally occurring amino acid
LKELAXS

SEQ ID NO: 18 - protein, 7 aa, Artificial Sequence
  Synthetic Peptide
  misc_feature (6)..(6): Xaa can be any naturally occurring amino acid
FKEIAXA

SEQ ID NO: 19 - protein, 7 aa, Artificial Sequence
  Synthetic Peptide
  misc_feature (6)..(6): Xaa can be any naturally occurring amino acid
LKELAXA

SEQ ID NO: 20 - protein, 30 aa, Artificial Sequence
  Synthetic Peptide
GKIEQILQKIEKILQKIEWILQKIEQILQG

SEQ ID NO: 21 - protein, 30 aa, Artificial Sequence
  Synthetic Peptide
GELKAIAQELKAIAKELKAIAWELKAIAQG

SEQ ID NO: 22 - protein, 30 aa, Artificial Sequence
  Synthetic Peptide
GEIAKSLKEIAKSLKEIAWSLKEIAKSLKG

SEQ ID NO: 23 - protein, 30 aa, Artificial Sequence
  Synthetic Peptide
GEIAQALREIAKALREIAWALREIAQALRG

SEQ ID NO: 24 - protein, 30 aa, Artificial Sequence
  Synthetic Peptide
GEIAKSLKEKAKSLKEIAWSLKEIAKSLKG

SEQ ID NO: 25 - protein, 30 aa, Artificial Sequence
  Synthetic Peptide
GEIAQALREIAKALREKAWALREIAQALRG

SEQ ID NO: 26 - protein, 30 aa, Artificial Sequence
  Synthetic Peptide
GEIAKALREIAKALREIAWALREDAKALRG

SEQ ID NO: 27 - protein, 30 aa, Artificial Sequence
  Synthetic Peptide
GEIAQALREIAKALREIAWALREKAQALRG

SEQ ID NO: 28 - protein, 30 aa, Artificial Sequence
  Synthetic Peptide
GEIAKALREIAKALREIAWALREEAKALRG

SEQ ID NO: 29 - protein, 30 aa, Artificial Sequence
  Synthetic Peptide
GEVAQAIKEVAKAIKEVAWAIKEVAQAIKG

SEQ ID NO: 30 - protein, 30 aa, Artificial Sequence
  Synthetic Peptide
GEIAQAIKEIAKAIKEIAWAIKEIAQAIKG

SEQ ID NO: 31 - protein, 30 aa, Artificial Sequence
  Synthetic Peptide
GEIAQAVKEIAKAVKEIAWAVKEIAQAVKG

SEQ ID NO: 32 - protein, 30 aa, Artificial Sequence
  Synthetic Peptide
GEVAQAVKEVAKAVKEVAWAVKEVAQAVKG

SEQ ID NO: 33 - protein, 30 aa, Artificial Sequence
  Synthetic Peptide
GEVAQALKEVAKALKEVAWALKEVAQALKG

SEQ ID NO: 34 - protein, 30 aa, Artificial Sequence
  Synthetic Peptide
GELAQAVKELAKAVKELAWAVKELAQAVKG

SEQ ID NO: 35 - protein, 30 aa, Artificial Sequence
  Synthetic Peptide
GELAQSIKELAKSIKELAWSIKELAQSIKG

SEQ ID NO: 36 - protein, 30 aa, Artificial Sequence
  Synthetic Peptide
GELSQAIKELSKAIKELSWAIKELSQAIKG

SEQ ID NO: 37 - protein, 30 aa, Artificial Sequence
  Synthetic Peptide
GELAQSIKELAKSIKEEAWSIKELAQSIKG

SEQ ID NO: 38 - protein, 30 aa, Artificial Sequence
  Synthetic Peptide
GELAQALKELAKALKELAWALKELAQALKG

SEQ ID NO: 39 - protein, 30 aa, Artificial Sequence [sequence omitted]

SEQ ID NO: 40 - protein, 30 aa, Artificial Sequence [sequence omitted]

SEQ ID NO: 41 - protein, 30 aa, Artificial Sequence
  Artificial Peptide
GELAQALKELAKALKEEAWALKELAQALKG

SEQ ID NO: 42 - protein, 30 aa, Artificial Sequence
  Artificial Peptide
GEIAQAFKEIAKAFKEIAWAFKEIAQAFKG

SEQ ID NO: 43 - protein, 30 aa, Artificial Sequence
  Artificial Peptide
GEIAQAMKEIAKAMKEIAWAMKEIAQAMKG

SEQ ID NO: 44 - protein, 30 aa, Artificial Sequence
  Artificial Peptide
GEIAQALKEIAKALKECAWALKEIAQALKG

SEQ ID NO: 45 - protein, 30 aa, Artificial Sequence
  Synthetic Peptide
  MISC_FEATURE (2)..(29): D-amino acid residue
GEVAQAVKEVAKAVKEVAWAVKEVAQAKVG

SEQ ID NO: 46 - protein, 30 aa, Artificial Sequence; identical to SEQ ID NO: 21
  Synthetic Peptide
  MOD_RES (24)..(24): Nle

SEQ ID NO: 47 - protein, 30 aa, Artificial Sequence; identical to SEQ ID NO: 23
  Synthetic Peptide
  MOD_RES (28)..(28): Nle

SEQ ID NO: 48 - protein, 30 aa, Artificial Sequence; identical to SEQ ID NO: 23
  Synthetic Peptide
  MISC_FEATURE (7)..(7): dehydroleucine
  MISC_FEATURE (14)..(14): dehydroleucine
  MISC_FEATURE (21)..(21): dehydroleucine
  MISC_FEATURE (28)..(28): dehydroleucine